(19) United States
(12) Patent Application Publication
(10) Pub. No.: US 2025/0025442 A1
(43) Pub. Date: Jan. 23, 2025

(54) BCAA-Lowering Compounds for Prevention and/or Treatment of Alzheimer's Disease and Related Disorders

(71) Applicant: Texas Tech University System, Lubbock, TX (US)

(72) Inventor: Andrew C. Shin, Lubbock, TX (US)

(21) Appl. No.: 18/256,449

(22) PCT Filed: Nov. 23, 2021
(86) PCT No.: PCT/US2021/060448

§ 371 Date: Jun. 8, 2023

Related U.S. Application Data
(60) Provisional application No. 63/123,886, filed on Dec. 10, 2020.

(51) Int. Cl. A61K 31/381; A61K 31/4245; A61P 25/28; G01N 33/68

(52) U.S. Cl. CPC A61K 31/381; A61K 31/4245; A61P 25/28; G01N 33/6812; G01N 2800/2821

(57) ABSTRACT

The present invention includes compositions and methods of treating CNS-related conditions, comprising: administering an effective amount of a compound that lowers the levels of one or more branched-chain amino acids (BCAAs) or metabolites thereof, or any pharmaceutically acceptable salt thereof, wherein the CNS-related conditions is selected from the group consisting of Alzheimer's disease and any related forms of dementia including vascular dementia, Lewy body dementia, frontotemporal dementia, Creutzfeldt-Jacob disease, Wernicke-Korsakoff disease, and Huntington's disease, Multiple sclerosis, Parkinson's disease, autism, Amyotrophic lateral sclerosis (ALS), Hereditary diffuse leukoencephalopathy with spheroids (HDLS) and epilepsy.

### STATEMENT OF FEDERALLY FUNDED RESEARCH

This invention was made with government support under 1R21AG069140-01 awarded by the National Institutes of Health/NSF/DARPA. The government has certain rights in the invention.

### CROSS-REFERENCE TO RELATED APPLICATIONS

None.

### TECHNICAL FIELD OF THE INVENTION

The present invention relates in general to the field of prevention and/or treatment of Alzheimer's disease and related disorders, and more particularly, to the use of BCAA-lowering compounds for prevention and/or treatment of Alzheimer's disease and related disorders.

### INCORPORATION-BY-REFERENCE OF MATERIALS FILED ON COMPACT DISC

None.

### BACKGROUND OF THE INVENTION

Without limiting the scope of the invention, its background is described in connection with Alzheimer's disease.

Alzheimer's disease (AD) is an irreversible neurodegenerative disorder ranking as the sixth leading cause of mortality in the US although more recent estimates suggest that the number is underestimated and it actually comes at third just after heart disease and cancer^{21,22}. Unfortunately, no cure or effective treatments exist currently that slow down the progression of AD. Without a specific biomarker that can predict the early onset of AD today we are mainly relying on several diagnostic tools to detect AD. NMR techniques such as PET imaging has been widely used to detect amyloid beta (AB) aggregates tau deposits in the brain (CNS) for AD diagnosis^{24,25}. However, injection of radioactive tracers, the need of trained physicians/technicians, uncomfortable procedure, and expensive cost remain as important limitations. Several diagnostic biomarkers can be obtained from the cerebrospinal fluid (CSF) that are both highly sensitive and specific^{25,28}, however the procedure involves an invasive lumbar puncture hat can cause nausea and severe backache in older patients. Clearly, ideal biomarkers would have not only high validation of diagnosis/prediction, but also only require a minimally invasive and risky technique such as those that can be obtained from blood. Several plasma/serum inflammatory cytokines²⁹⁻³² Aβ peptides^{28,33}, and mi-RNAs³⁴⁻³⁶ have shown promising results for detection of AD, although inconsistent findings and the lack of reproducibility far limit the applicability of these potential biomarkers. Collectively, identification of reliable circulating biomarkers that are minimally invasive and patient-friendly remain as a great interest and priority in the field.

Alzheimer's disease (AD) affects over 5 million individuals in the US without any cure or effective treatments. Further, there is no reliable biomarker today that can help predict individual risk for AD, thus raising the national urgency to bridge the major gap in strategies to prevent and/or treat AD.

Thus, what is needed are novel methods for preventing and treating AD.

### SUMMARY OF THE INVENTION

In one embodiment, the present invention includes a method of treating CNS-related conditions, comprising: administering an effective amount of a compound that lowers the levels of one or more branched-chain amino acids (BCAAs) or metabolites thereof, or any pharmaceutically acceptable salt thereof, wherein the CNS-related conditions is selected from the group consisting of Alzheimer's disease and dementia selected from the group consisting of vascular dementia, Lewy body dementia, frontotemporal dementia, Creutzfeldt-Jacob disease, Wernicke-Korsakoff disease, and Huntington's disease, Multiple sclerosis, Parkinson's disease, autism, Amyotrophic lateral sclerosis (ALS), Hereditary diffuse leukoencephalopathy with spheroids (HDLS) and epilepsy, neuropsychiatric disorders such as all anxiety disorders including, but not limited to, generalized anxiety disorder, social anxiety disorder, specific phobias and separation anxiety disorder, and a collection of depression disorders including, but not limited to, clinical depression (major depression), bipolar depression, persistent depressive disorder (dysthymia), seasonal affective disorder, atypical depression, treatment-resistant depression, psychotic depression, postpartum depression, premenstrual dysphoric disorder, and situational depression (stress response syndrome). In one aspect, the compound is a (S)-α-chloro-phenylpropionic acid and is administered in an amount within a range of from 10-200 mg/kg and is provided daily. In another aspect, the compound is a BT2 compound and is provided at administered in an amount within a range of from 10-100 mg/kg/day and is provided daily. In another aspect, the BT2 compounds is selected from at least one of: BT2 (3,6-dichlorobenzo[b]thiophene-2-carboxylic acid); BT2F (3-chloro-6-fluorobenzo[b]thiophene-2-carboxylic acid); or BT3 (N-(4-acetamido-1,2,5-oxadiazol-3-yl)-3,6-dichlorobenzo[b]thiophene-2-carboxamide). In another aspect, the compound is a benzothiopene-2-carboxylic acid (BT2) compound and is provided at administered in an amount within a range of from 10-100 mg/kg/day and is provided daily. In another aspect, the compound is administered using a method selected from the group consisting of oral, intravenous, subcutaneous, intranasal, pulmonary, intracranial, intracerebroventricular, topical, enteral, parenteral, or rectal. In another aspect, the compound is administered in a formulation comprising a carrier, the carrier being selected from the group consisting of: lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil. In another aspect, the CNS-related condition treated by the compound is Alzheimer's Disease. In another aspect, the method further comprises obtaining a biological sample from the patient with the CNS-related condition and determining if the biological sample has an increase in BCAAs or metabolites thereof when compared to a sample from a subject without the CNS-related condition.

In another embodiment, the present invention includes a method of identifying and treating CNS-related conditions, comprising: obtaining a biological sample from the patient with the CNS-related condition and determining if the biological sample has an increase in one or more branched-chain amino acids (BCAAs) or metabolites thereof, when compared to a sample from a subject without the CNS-related condition; and administering an effective amount of a compound that lowers the levels of one or more branched-chain amino acids (BCAAs) or metabolites thereof, or any pharmaceutically acceptable salt thereof, wherein the CNS-related conditions is selected from the group consisting of Alzheimer's disease and dementia selected from the group consisting of vascular dementia, Lewy body dementia, frontotemporal dementia, Creutzfeldt-Jacob disease, Wernicke-Korsakoff disease, and Huntington's disease, Multiple sclerosis, Parkinson's disease, autism, Amyotrophic lateral sclerosis (ALS), Hereditary diffuse leukoencephalopathy with spheroids (HDLS) and epilepsy, neuropsychiatric disorders such as all anxiety disorders including, but not limited to, generalized anxiety disorder, social anxiety disorder, specific phobias and separation anxiety disorder, and a collection of depression disorders including, but not limited to, clinical depression (major depression), bipolar depression, persistent depressive disorder (dysthymia), seasonal affective disorder, atypical depression, treatment-resistant depression, psychotic depression, postpartum depression, premenstrual dysphoric disorder, and situational depression (stress response syndrome). In one aspect, the compound is a (S)-α-chloro-phenylpropionic acid and is administered in an amount within a range of from 10-200 mg/kg and is provided daily. In another aspect, the compound is a benzothiopene-2-carboxylic acid (BT2) compound and is provided at administered in an amount within a range of from 10-100 mg/kg/day and is provided daily. In another aspect, the BT2 compounds is selected from at least one of: BT2 (3,6-dichlorobenzo[b]thiophene-2-carboxylic acid); BT2F (3-chloro-6-fluorobenzo[b]thiophene-2-carboxylic acid); or BT3 (N-(4-acetamido-1,2,5-oxadiazol-3-yl)-3,6-dichlorobenzo[b]thiophene-2-carboxamide). In another aspect, the compound is a BT2 compound and is provided at administered in an amount within a range of from 10-100 mg/kg/day and is provided daily. In another aspect, the compound is administered using a method selected from the group consisting of oral, intravenous, subcutaneous, intranasal, pulmonary, intracranial, intracerebroventricular, topical, enteral, parenteral, or rectal. In another aspect, the compound is administered in a formulation comprising a carrier, the carrier being selected from the group consisting of: lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil. In another aspect, the CNS-related condition treated by the compound is Alzheimer's Disease. In another aspect, the method further comprises obtaining one or more additional biological samples at a different time from the patient with the CNS-related condition after treatment with the composition that lowers one or more BCAAs or metabolites thereof, and determining if the biological sample has an decrease in BCAAs when compared to a prior sample from the subject with the CNS-related condition.

In another embodiment, the present invention includes a method for treating a patient with a CNS-related condition that comprises an increase in one or more branched-chain amino acids (BCAAs) or metabolites thereof, the method comprising the steps of: performing or having performed an assay from a biological sample from the patient with the CNS-related condition to determine if the biological sample has an increase in one or more branched-chain amino acids (BCAAs) or metabolites thereof, when compared to a sample from a subject without the CNS-related condition; and if the biological sample from the patient with the CNS-related condition has an increase in the one or more BCAAs or metabolites thereof, then: treating the patient with an effective amount of a compound that lowers the levels of one or more branched-chain amino acids (BCAAs) or metabolites thereof, or any pharmaceutically acceptable salt thereof, wherein the CNS-related conditions is selected from the group consisting of Alzheimer's disease and dementia selected from the group consisting of vascular dementia, Lewy body dementia, frontotemporal dementia, Creutzfeldt-Jacob disease, Wernicke-Korsakoff disease, and Huntington's disease, Multiple sclerosis, Parkinson's disease, autism, Amyotrophic lateral sclerosis (ALS), Hereditary diffuse leukoencephalopathy with spheroids (HDLS) and epilepsy, neuropsychiatric disorders such as all anxiety disorders including, but not limited to, generalized anxiety disorder, social anxiety disorder, specific phobias and separation anxiety disorder, and a collection of depression disorders including, but not limited to, clinical depression (major depression), bipolar depression, persistent depressive disorder (dysthymia), seasonal affective disorder, atypical depression, treatment-resistant depression, psychotic depression, postpartum depression, premenstrual dysphoric disorder, and situational depression (stress response syndrome). In one aspect, the compound is a (S)-α-chloro-phenylpropionic acid and is administered in an amount within a range of from 10-200 mg/kg and is provided daily. In another aspect, the compound is a benzothiopene-2-carboxylic acid (BT2) compound and is provided at administered in an amount within a range of from 10-100 mg/kg/day and is provided daily. In another aspect, the BT2 compounds is selected from at least one of: BT2 (3,6-dichlorobenzo[b]thiophene-2-carboxylic acid); BT2F (3-chloro-6-fluorobenzo[b]thiophene-2-carboxylic acid); or BT3 (N-(4-acetamido-1,2,5-oxadiazol-3-yl)-3,6-dichlorobenzo[b]thiophene-2-carboxamide). In another aspect, the compound is a BT2 compound and is provided at administered in an amount within a range of from 10-100 mg/kg/day and is provided daily. In another aspect, the compound is administered using a method selected from the group consisting of oral, intravenous, subcutaneous, intranasal, pulmonary, intracranial, intracerebroventricular, topical, enteral, parenteral, or rectal.

In another embodiment, the present invention includes a method for diagnosing a patient with a CNS-related condition related to increases in branched-chain amino acids (BCAAs) or metabolites thereof, the method comprising: performing or having performed an assay from a biological sample from the patient with the CNS-related condition to determine if the biological sample has an increase in one or more branched-chain amino acids (BCAAs) or metabolites thereof, when compared to a sample from a subject without the CNS-related condition, wherein the BCAA is selected from at least one of valine, leucine, and isoleucine, or metabolites thereof. In one aspect, the biological sample is selected from blood, plasma, serum, tear, sweat, or sputum. In another aspect, the CNS-related condition is selected from at least one of: Alzheimer's disease and dementia selected from the group consisting of vascular dementia, Lewy body dementia, frontotemporal dementia, Creutzfeldt-Jacob disease, Wernicke-Korsakoff disease, and Huntington's disease, Multiple sclerosis, Parkinson's disease, autism, Amyotrophic lateral sclerosis (ALS), Hereditary diffuse leukoencephalopathy with spheroids (HDLS) and epilepsy, neuropsychiatric disorders such as all anxiety disorders including, but not limited to, generalized anxiety disorder, social anxiety disorder, specific phobias and separation anxiety disorder, and a collection of depression disorders including, but not limited to, clinical depression (major depression), bipolar depression, persistent depressive disorder (dysthymia), seasonal affective disorder, atypical depression, treatment-resistant depression, psychotic depression, postpartum depression, premenstrual dysphoric disorder, and situational depression (stress response syndrome).

### BRIEF DESCRIPTION OF THE DRAWINGS

For a more complete understanding of the features and advantages of the present invention, reference is now made to the detailed description of the invention along with the accompanying figures and in which:

FIGS. 1A to 1F show individuals with Alzheimer's disease (AD) show high circulating BCAAs and their intermediates beyond the effect of Type 2 diabetes. Plasma samples from healthy controls, patients with AD, diabetes, or with diabetes+AD after overnight fasting were analyzed using LC/MS. (FIG. 1A) Healthy vs. AD; (FIG. 1B) Healthy vs. Diabetes; (FIG. 1C) Diabetes vs. Diabetes+AD. Data shown are fold changes normalized to the mean of healthy controls. Analyzed by student's t-test. *p<0.05D) Composite predictor “BCAA” significantly distinguished AD patients from healthy subjects in a predictive model. Iso: Isoleucine; HMG:3-methylglutaryl (metabolite of leucine); KIV: ketoisovalerate (metabolite of valine); KIC: ketoisocaproate (metabolite of leucine); MMSA: Methyl-melonatesemialdehyde (metabolite of valine); Phe: Phenylalanine; 2-MPA: 2-methylpropanoicacid (metabolite of valine); 3-HP: 3-OH-Propanoate (metabolite of valine). (FIG. 1D) Correlation analysis between serum 2-MBC levels and CDR scores or FIG. 1E) MMSE scores; FIG. 1F) Correlation analysis between serum sotolone, a metabolite of isoleucine, and MMSE scores. Iso: Isoleucine; HMG: 3-methylglutaryl (metabolite of leucine); KIV: ketoisovalerate (metabolite of valine); KIC: ketoisocaproate (metabolite of leucine); MMSA: Methyl-melonatesemialdehyde (metabolite of valine); Phe: Phenylalanine; 2-MPA: 2-methylpropanoic acid (metabolite of valine); 3-HP: 3-OH-Propanoate (metabolite of valine); 2-MBC: 2-methylbutyrylcarnitine (metabolite of isoleucine); CDR: Clinical Dementia Rating; MMSE: Mini-Mental State Examination.

FIGS. 2A to 2F shows BCAA metabolism is impaired in transgenic AD mice. (FIG. 2A) Fasting plasma BCAAs are elevated in 9 month-old AD mice compared to WT littermates. (FIG. 2B) Western blots showing liver protein abundance of BCKDH, the rate-limiting enzyme in BCAA breakdown; phosphorylated, inactive state of BCKDH (pBCKDH); BCKDH kinase, enzyme that phosphorylates BCKDH; and BCAT, an enzyme involved in the reversible first step in BCAA degradation. (FIG. 2C) pBCKDH is significantly increased in AD mice, resulting in higher inactivity index (pBCKDH/BCKDHratio). (FIG. 2D) Increased hepatic BCKDH kinase in AD mice compared to WTs. (FIG. 2E) Gene analysis via RT-qPCR in liver shows a significant increase in BCKDH kinase which is in agreement with the protein data. (FIG. 2F) Validation of APP/PS1 transcripts in AD mice by PCR.NC-negative control; PC-positive control. *p<0.05 compared to WT littermates. Phosph-BCKDH phosphatase; Kinase-BCKDH kinase

FIGS. 3A to 3H. BCAA treatment in differentiated HT-22 hippocampal neurons dose-dependently alters genes similar to what occurs in AD brain. FIG. 3A. Downregulation of genes involved in mitochondrial biogenesis, fusion, and synapse formation following BCAA addition in media for 24 h. FIG. 3B. Western blots showing pGSK3β, total GSK3β, pTau 396, total Tau. FIG. 3C. Significantly lower pGSK3β in BCAA-treated groups, indicating more activation of GSK3β. This probably leads to FIG. 3D significantly elevated pTau in BCAA-treated groups, similar to that observed in AD brain. N=6/group; *p<0.05 compared to Control. A significant downregulation of genes responsible for mitochondrial biogenesis and fusion as well as synapse formation was verified in an independent cohort (FIG. 3E). BCAA supplementation for 24 h was enough to dramatically lower mRNAs that encode enzymes involved in glycolysis, including the rate-limiting enzymes hexokinase and pyruvate kinase (FIG. 3F). In agreement with BCAA-induced neuronal dysfunction, mRNA abundance of pro-inflammatory mediators TNF-α and IL-6 was markedly increased compared to vehicle-treated HT-22 neurons (FIGS. 3G, 3H).

FIGS. 4A to 4H show that APP/PS1 mice displayed higher plasma BCAA levels compared to WTs before dietary treatment (FIG. 4A), shows APP_{swe} mouse model. FIGS. 4B and 4C show that diet change did not affect body weight or food intake for the entire treatment duration. FIG. 4D shows there was no difference in blood glucose between groups at baseline, after two months a significantly increased blood glucose was observed in regular chow-fed APP/PS1 mice compared to that in WT controls, as well as their own baseline. FIG. 4D shows that BCAA restriction was able to prevent the rise of blood glucose in APP/PS1 mice. FIG. 4E shows the Y-maze widely used behavioral test used to assess working memory. FIG. 4F shows that APP/PS1 mice did not show any differences in spontaneous alternation compared to WTs at baseline, most likely because they were not old enough to show any cognitive dysfunction (ex. spatial learning) which usually occurs between 12-13 month of age. FIGS. 4G to 4J show behavioral outcome induced by BCAA restriction is partly due to changes in the mobility or distraction of the animals, total distance (FIG. 4G), freezing time (FIG. 4H), number of entries (FIG. 4I), and mean speed (FIG. 4J).

FIGS. 5A to 5N show BCAA-restricted APP/PS1 mice had a significantly lower ratio of pBCKDH to BCKDH in liver compared to regular chow-fed APP/PS1 mice. (FIGS. 5A and 5B) indicating enhanced hepatic BCAA catabolism that would likely lead to lower circulating BCAAs. FIGS. 5C, 5D shows the inactivity index and BCKDH kinase protein expression were not different in the cortex across groups. FIG. 5E shows APP/PS1 mice fed BCAA-restricted diet displayed significantly lower Aβ-42 levels compared to the group on a regular chow diet. FIG. 5F shows that APP/PS1 controls showed a markedly decreased protein expression of insulin-degrading enzyme (IDE), the enzyme known to break down amyloid peptide, BCAA restriction completely reversed it. FIGS. 5G-J shows that limiting BCAA intake was able to nullify the increased, phosphorylated state of Tau at Thr205, but not at Ser202 or 396, in APP/PS1 mice compared to those fed a regular chow diet. FIG. 5K shows the effects of BCAA supplementation on HT-22 hippocampal neurons, PSD95 protein was not increased in BCAA-restricted APP/PS1 mice compared to chow-fed counterparts or WTs. FIG. 5L shows APP/PS1 mice fed BCAA-restricted diet displayed a trend of decreased pro-inflammatory markers such as TNF-α and IL-6, without any changes in Aβ-degrading enzymes including IDE and neprilysin. FIGS. 5M and 5N show that regular chow-fed APP/PS1 mice showed reduced levels of NE, DA and its metabolite DOPAC, and 5-HT in the cortex and hippocampus compared to WTs, but BCAA restriction for two months was able to completely normalize the NT concentrations in both brain regions, most notably DA and DOPAC.

FIGS. 6A to 6M show that transgenic mice develop amyloid plaques and neuroinflammation at as early as 1.5-2 months of age. FIG. 6A shows that body weight FIG. 6B food intake were not different between WT and 5xFAD mice regardless of treatments. FIG. 6C shows elevated blood glucose compared to WTs before treatment (p<0.05; FIG. 6C). FIG. 6D shows the results from APP_{swe} and APP/PS1 mouse experiments, 5xFAD mice showed significantly higher plasma BCAA levels compared to WT mice at baseline). FIG. 6C shows APP/PS1 mice fed BCAA-restriction diet, BT2-treated 5xFAD mice were able to markedly decrease blood glucose compared to vehicle-treated 5xFAD mice. No change was seen within WT groups. FIG. 6E shows amyloid and Tau pathology in the cortex. FIGS. 6F and 6G shows decreased amyloid burden is most likely due to elevated Aβ-degrading enzyme IDE by BT2 (70%)(FIG. 6H) shows expression data). FIG. 6L shows reduced AD-related pathology, Tau-phosphorylating and pro-inflammatory enzyme GSK3β was significantly reduced in the cortex after BT2 treatment. In support of reduced AD-related pathology, Tau-phosphorylating and pro-inflammatory enzyme GSK3β was significantly reduced in the cortex after BT2 treatment (FIG. 6I). FIG. 6Js show that key mediators of ER stress (PERK) and inflammation (NF-κB) was decreased in BT2-treated 5xFAD mice, and more interestingly, BACE1 (a.k.a. β-secretase) that is responsible for cleaving APP to promote Aβ-42 production was markedly lowered in these mice. FIG. 6K shows that 5xFAD control mice have a significant reduction of NE compared to WT controls in the hippocampus, but NE level is fully restored in BT2-treated 5xFAD mice. FIG. 6L shows results for DA (180%) and its metabolite DOPAC (300%) in the cortex of BT2-treated 5xFAD mice. FIG. 6M shows protein expression of tyrosine hydroxylase (TH), the rate-limiting enzyme for synthesis of catecholamines including NE and DA. While there was no difference between WT groups, BT2 treatment increased TH level by nearly 400% in 5xFAD mice (p=0.07;).

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

The present inventors identified a class of existing compounds to prevent or delay the progression of Alzheimer's disease (AD). There are several important aspects of the present invention. (1) These compounds' mechanism of action has been already tested and validated by many laboratories and are easily available. Since current drugs are not effective at treating AD, this is a huge advantage because pharmaceutical companies do not need to spend millions of dollars just to develop an effective compound, and instead can take advantage of these validated compounds providing an immediate benefit to patients. (2) Another innovative element lies in the process for identifying novel uses for these compounds. These compounds have recently been shown to lower branched-chain amino acids (BCAAs) in rodents and alleviate insulin resistance and restore heart remodeling. The present inventor shows herein that circulating BCAAs are higher in both individuals and animal model of AD, and BCAA supplementation induces features of AD pathology, suggesting that the BCAA-lowering compounds can potentially ameliorate AD pathology and symptoms. (3) Development/refining of these compounds to test its efficacy and safety can be conducted using well-known neuroscience and biochemistry methods and models. Rapid, successful testing in both animals and humans allows these compounds to immediately benefit patients.

Alzheimer's disease (AD) is the third leading cause of mortality in the US and the number of cases is expected to exponentially increase highlighting the need for developing an effective treatment. The annual healthcare expenditure is nearly $300 billion and is a huge burden on the nation's healthcare system. It is estimated that the number of people with AD and other forms of dementia will grow to 13.9 million by 2060. The quality of life of AD patients is severely impacted by memory loss; they are unable to care for themselves and rely on assisted living and the support of unpaid caregivers. It is also important to tend to the well-being and needs of the caregivers. The development of drugs that improve the control of symptoms will be very beneficial for at-risk patients and caregivers to plan for specialized professional care. Moreover, given that AD shares similar pathophysiological abnormalities with other neurodegenerative diseases such as Parkinson's disease, Huntington's disease, autism, and other prevalent chronic disorders such as obesity, Type 2 diabetes and cardiovascular diseases, the proposed compounds may potentially be broadly used for treating these different disorders alone or in conjunction with current existing therapies. The present invention can be used to treat Alzheimer's disease and dementia selected from the group consisting of vascular dementia, Lewy body dementia, frontotemporal dementia, Creutzfeldt-Jacob disease, Wernicke-Korsakoff disease, and Huntington's disease, Multiple sclerosis, Parkinson's disease, autism, Amyotrophic lateral sclerosis (ALS), Hereditary diffuse leukoencephalopathy with spheroids (HDLS) and epilepsy, neuropsychiatric disorders, generalized anxiety disorder, social anxiety disorder, specific phobias and separation anxiety disorder, depression disorders, clinical depression (major depression), bipolar depression, persistent depressive disorder (dysthymia), seasonal affective disorder, atypical depression, treatment-resistant depression, psychotic depression, postpartum depression, premenstrual dysphoric disorder, and situational depression (stress response syndrome).

A dosage unit for use of the compounds or agents that reduce a level of branched-chain amino acids (BCAAs) of the present invention, may be a single compound or mixtures thereof with other compounds. The compounds may be mixed together, form ionic or even covalent bonds. The BCAA-reducing agents of the present invention may be administered in oral, intravenous (bolus or infusion), intraperitoneal, subcutaneous, or intramuscular form, all using dosage forms well known to those of ordinary skill in the pharmaceutical arts. Depending on the particular location or method of delivery, different dosage forms, e.g., tablets, capsules, pills, powders, granules, elixirs, tinctures, suspensions, syrups, and emulsions may be used to provide the reduction in the levels of BCAAs of the present invention to a patient in need of therapy for CNS-related conditions, such as Alzheimer's Disease and other neurological conditions caused by an increase in BCAAs. The BCAA reducing agents may also be administered as any one of known salt forms.

BCAA-reducing agents re typically administered in admixture with suitable pharmaceutical salts, buffers, diluents, extenders, excipients and/or carriers (collectively referred to herein as a pharmaceutically acceptable carrier or carrier materials) selected based on the intended form of administration and as consistent with conventional pharmaceutical practices. Depending on the best location for administration, the BCAA reducing agents may be formulated to provide, e.g., maximum and/or consistent dosing for the particular form for oral, rectal, topical, intravenous injection or parenteral administration. While each of the BCAA reducing agents may be administered alone, it will generally be provided in a stable salt form mixed with a pharmaceutically acceptable carrier. The carrier may be solid or liquid, depending on the type and/or location of administration selected.

The following are non-limiting examples of lower BCAA lowering agents for use with the present invention. (S)-CPP: (S)-α-chloro-phenylpropionic acid. All in vitro pharmacokinetics (ex. affinity binding, binding site, functional activity) examined and demonstrated (Tso S C et al., PNAS, 2013). Half-life of ˜7 hours in hepatocytes, and 0.113 ml/min clearance rate in CD-1 mice during in vivo pharmacokinetics study (meaning it stays long enough in the body). Shown to effectively inhibit BCKDH kinase (BCKDH suppressor), thereby activating BCKDH to break down BCAAs in liver, muscle, kidney, and heart, and thus lowering plasma BCAAs in mice (Tso S C et al., PNAS, 2013). A commonly used dose is: 40-160 mg/kg/day intraperitoneal (ip) injection for 3-8 weeks, but doses of 1-250 mg can be used, e.g., 10, 20, 30, 40, 50, 60, 70, 80, 90, 100, 110, 120, 125, 130, 140, 150, 160, 170, 175, 180, 190 or 200 mg can be used. Dose and/or duration are tested for safety and efficacy in Alzheimer's mice and then humans. BT2 compounds: BT2 (3,6-dichlorobenzo[b]thiophene-2-carboxylic acid); BT2F (3-chloro-6-fluorobenzo[b]thiophene-2-carboxylic acid); BT3 (N-(4-acetamido-1,2,5-oxadiazol-3-yl)-3,6-dichlorobenzo[b]thiophene-2-carboxamide). All in vitro pharmacokinetics (ex. affinity binding, binding site, functional activity) examined and demonstrated (Tso S C et al. J Biol Chem, 2014). Half-life of >4 hours in vitro and ˜12 hours when injected ip in CD-1 mice during in vivo pharmacokinetics study (meaning it stays long enough in the body). Shown to effectively inhibit BCKDH kinase (BCKDH suppressor), thereby activating BCKDH to break down BCAAs in liver, muscle, kidney, and heart, and thus lowering plasma BCAAs in mice (Tso S C et al., J Biol Chem, 2014). A commonly used dose is: 20-40 mg/kg/day ip injection or oral gavage for 3-8 week, but doses of 1-100 mg can be used, e.g., 1, 2, 3, 4, 5, 6, 7, 8, 9, 10, 20, 25, 30, 40, 50, 60, 70, 75, 80, 90 or 100 mg can be used. Dose and/or duration are tested for safety and efficacy in Alzheimer's mice and then humans. BT2 is used for Lowering plasma BCAAs and their metabolites to alleviate obesity-related insulin resistance. Lowering heart BCAAs and their metabolites to restore heart function or remodeling after experimental heart failure.

Techniques and compositions for making useful dosage forms using the present invention are described in one or more of the following references: Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2007; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins, 2000, and updates thereto; Martindale, The Extra Pharmacopoeia, Thirty-Second Edition (The Pharmaceutical Press, London, 1999); relevant portions incorporated herein by reference.

For example, the BCAA-reducing agents may be included in a tablet. Tablets may contain, e.g., suitable binders, lubricants, disintegrating agents, coloring agents, flavoring agents, flow-inducing agents and/or melting agents. For example, oral administration may be in a dosage unit form of a tablet, gelcap, caplet or capsule, the active drug component being combined with an non-toxic, pharmaceutically acceptable, inert carrier such as lactose, gelatin, agar, starch, sucrose, glucose, methyl cellulose, magnesium stearate, dicalcium phosphate, calcium sulfate, mannitol, sorbitol, mixtures thereof, and the like. Suitable binders for use with the present invention include: starch, gelatin, natural sugars (e.g., glucose or beta-lactose), corn sweeteners, natural and synthetic gums (e.g., acacia, tragacanth or sodium alginate), carboxymethylcellulose, polyethylene glycol, waxes, and the like. Lubricants for use with the invention may include: sodium oleate, sodium stearate, magnesium stearate, sodium benzoate, sodium acetate, sodium chloride, mixtures thereof, and the like. Disintegrators may include: starch, methyl cellulose, agar, bentonite, xanthan gum, mixtures thereof, and the like.

BCAA-reducing agents may be administered in the form of liposome delivery systems, e.g., small unilamellar vesicles, large unilamellar vesicles, and multilamellar vesicles, whether charged or uncharged. Liposomes may include one or more: phospholipids (e.g., cholesterol), stearylamine and/or phosphatidylcholines, mixtures thereof, and the like.

BCAA-reducing agents may also be coupled to one or more soluble, biodegradable, bioacceptable polymers as drug carriers or as a prodrug. Such polymers may include: polyvinylpyrrolidone, pyran copolymer, polyhydroxylpropylmethacrylamide-phenol, polyhydroxyethylasparta-midephenol, or polyethyleneoxide-polylysine substituted with palmitoyl residues, mixtures thereof, and the like. Furthermore, the BCAA reducing agents may be coupled one or more biodegradable polymers to achieve controlled release of the BCAA reducing agents, biodegradable polymers for use with the present invention include: polylactic acid, polyglycolic acid, copolymers of polylactic and polyglycolic acid, polyepsilon caprolactone, polyhydroxy butyric acid, polyorthoesters, polyacetals, polydihydropyrans, polycyanoacylates, and crosslinked or amphipathic block copolymers of hydrogels, mixtures thereof, and the like.

In one embodiment, gelatin capsules (gelcaps) may include the BCAA-reducing agents and powdered carriers, such as lactose, starch, cellulose derivatives, magnesium stearate, stearic acid, and the like. Like diluents may be used to make compressed tablets. Both tablets and capsules may be manufactured as immediate-release, mixed-release or sustained-release formulations to provide for a range of release of medication over a period of minutes to hours. Compressed tablets may be sugar coated or film coated to mask any unpleasant taste and protect the tablet from the atmosphere. An enteric coating may be used to provide selective disintegration in, e.g., the gastrointestinal tract.

For oral administration in a liquid dosage form, the oral drug components may be combined with any oral, non-toxic, pharmaceutically acceptable inert carrier such as ethanol, glycerol, water, and the like. Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents, mixtures thereof, and the like.

Liquid dosage forms for oral administration may also include coloring and flavoring agents that increase patient acceptance and therefore compliance with a dosing regimen. In general, water, a suitable oil, saline, aqueous dextrose (e.g., glucose, lactose and related sugar solutions) and glycols (e.g., propylene glycol or polyethylene glycols) may be used as suitable carriers for parenteral solutions. Solutions for parenteral administration include generally, a water soluble salt of the active ingredient, suitable stabilizing agents, and if necessary, buffering salts. Antioxidizing agents such as sodium bisulfite, sodium sulfite and/or ascorbic acid, either alone or in combination, are suitable stabilizing agents. Citric acid and its salts and sodium EDTA may also be included to increase stability. In addition, parenteral solutions may include pharmaceutically acceptable preservatives, e.g., benzalkonium chloride, methyl- or propyl-paraben, and/or chlorobutanol. Suitable pharmaceutical carriers are described in Remington's Pharmaceutical Sciences, Mack Publishing Company, a standard reference text in this field, relevant portions incorporated herein by reference.

For direct delivery to the nasal passages, sinuses, mouth, throat, esophagus, trachea, lungs and alveoli, the BCAA reducing agents may also be delivered as an intranasal form via use of a suitable intranasal vehicle. For dermal and transdermal delivery, the BCAA reducing agents may be delivered using lotions, creams, oils, elixirs, serums, transdermal skin patches and the like, as are well known to those of ordinary skill in that art. Parenteral and intravenous forms may also include pharmaceutically acceptable salts and/or minerals and other materials to make them compatible with the type of injection or delivery system chosen, e.g., a buffered, isotonic solution. Examples of useful pharmaceutical dosage forms for administration of BCAA reducing agents may include the following forms.

Capsules. Capsules may be prepared by filling standard two-piece hard gelatin capsules each with 1 to 500 milligrams of powdered active ingredient, 5 to 150 milligrams of lactose, 5 to 50 milligrams of cellulose and 6 milligrams magnesium stearate.

Soft Gelatin Capsules. A mixture of active ingredient is dissolved in a digestible oil such as soybean oil, cottonseed oil or olive oil. The active ingredient is prepared and injected by using a positive displacement pump into gelatin to form soft gelatin capsules containing, e.g., 100-500 milligrams of the active ingredient. The capsules are washed and dried.

Tablets. A large number of tablets are prepared by conventional procedures so that the dosage unit was 1 to 500 milligrams of active ingredient, 0.2 milligrams of colloidal silicon dioxide, 5 milligrams of magnesium stearate, 50-275 milligrams of microcrystalline cellulose, 11 milligrams of starch and 98.8 milligrams of lactose. Appropriate coatings may be applied to increase palatability or delay absorption.

To provide an effervescent tablet appropriate amounts of, e.g., monosodium citrate and sodium bicarbonate, are blended together and then roller compacted, in the absence of water, to form flakes that are then crushed to give granulates. The granulates are then combined with the active ingredient, drug and/or salt thereof, conventional beading or filling agents and, optionally, sweeteners, flavors and lubricants.

Injectable solution. A parenteral composition suitable for administration by injection is prepared by stirring 1.5% by weight of active ingredient in deionized water and mixed with, e.g., up to 10% by volume propylene glycol and water. The solution is made isotonic with sodium chloride and sterilized using, e.g., ultrafiltration.

Suspension. An aqueous suspension is prepared for oral administration so that each 5 ml contain 1 to 500 mg of finely divided active ingredient, 200 mg of sodium carboxymethyl cellulose, 5 mg of sodium benzoate, 1.0 g of sorbitol solution, U.S.P., and 0.025 ml of vanillin.

For mini-tablets, the active ingredient is compressed into a hardness in the range 6 to 12 Kp. The hardness of the final tablets is influenced by the linear roller compaction strength used in preparing the granulates, which are influenced by the particle size of, e.g., the monosodium hydrogen carbonate and sodium hydrogen carbonate. For smaller particle sizes, a linear roller compaction strength of about 15 to 20 KN/cm may be used.

Kits. The present invention also includes pharmaceutical kits useful, for example, for the treatment of cancer, which comprise one or more containers containing a pharmaceutical composition comprising a therapeutically effective amount of BCAA-reducing agents. Such kits may further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, may also be included in the kit. It should be understood that although the specified materials and conditions are important in practicing the invention, unspecified materials and conditions are not excluded so long as they do not prevent the benefits of the invention from being realized.

Examples of suitable liquid dosage forms include solutions or suspensions in water, pharmaceutically acceptable fats and oils, alcohols or other organic solvents, including esters, emulsions, syrups or elixirs, suspensions, solutions and/or suspensions reconstituted from non-effervescent granules and effervescent preparations reconstituted from effervescent granules. Such liquid dosage forms may contain, for example, suitable solvents, preservatives, emulsifying agents, suspending agents, diluents, sweeteners, thickeners, and melting agents. Oral dosage forms optionally contain flavorants and coloring agents. Parenteral and intravenous forms may also include minerals and other materials to make them compatible with the type of injection or delivery system chosen.

As used herein, the term “chewable” refers to semi-soft, palatable and stable chewable treat without addition of water. It should be appreciated to the skilled artisan that a chewable composition will be stable and palatable, fast disintegrating, semi-soft medicated chewable tablets (treats) by extrusion without the addition of extraneous water. A soft, chewable tablet does not harden on storage and are resistant to microbial contamination. A semi-soft chewable contain a blend of any one or more of binders, flavours, palatability enhancers, humectants, disintegrating agents, non-aqueous solvents, and diluents that are plasticized with liquid plasticizers, such as glycols and polyols to make them ductile and extrudable. The chewable can be made by extrusion, e.g., including fats or lipids as plasticizers and binding agents, is manufactured in the absence of added water, uses plasticizers to replace water in extrudable matrices, contains humectants to maintain the extrudable chew in a pliant and soft state during its shelf life, or any combination thereof. The chewable form may be provided in conjunction with one or more flavorants and/or taste masking agents that improve the taste of the formulation greater than 10, 20, 30, 40, 50, 60, 70, 80, or 90%. The chewable can include the active agent and the ion exchange resin to enhance taste masking.

For topical administration, the composition can be incorporated into creams, ointments, gels, transdermal patches and the like. The composition can also be incorporated into medical dressings, for example wound dressings, e.g., woven (e.g., fabric) dressings or non-woven dressings (e.g., gels or dressings with a gel component). The use of alginate polymers in dressings is known, and such dressings, or indeed any dressings, may further incorporate the alginate oligomers of the invention.

Branched-chain amino acids (BCAAs) are essential amino acids that need to be obtained from food. They compete for same transporter with other aromatic amino acids that serve as the precursors of several critical neurotransmitters in the brain such as dopamine, serotonin, and norepinephrine¹, hence excess BCAAs may lead to profound imbalance of these key neurotransmitters. Moreover, BCAA overload has been shown to induce neural oxidative stress and apoptosis⁴⁻⁹, and mTOR hyperactivation commonly induced by BCAAs lead to insulin resistance in the brain¹⁰⁻¹¹. Importantly, all of these abnormalities represent the pathophysiological hallmarks of AD, making BCAAs a potentially significant contributor to the development of AD. Interestingly, BCAAs are recently implicated in insulin resistance/Type 2 diabetes (T2D)¹²⁻¹⁷. The present inventors determined if defective BCAA metabolism drives development or exacerbation of AD, however the role of BCAAs in AD pathogenesis is poorly understood.

### Example 1. Examine BCAA Metabolism in a Mouse Model of Alzheimer's Disease

Time-course study to determine plasma and brain BCAA profiles are associated with AD. Pathological features of AD such as phosphorylated Tau protein and AB aggregates in the neocortex and hippocampus and cognitive deficits will be assessed in a well-established APP/PS1 mouse model of AD.

Test if high plasma BCAAs and/or their metabolites in AD are due to impaired hepatic BCAA catabolism. He inventor has previously shown that insulin regulates plasma BCAAs by inducing hepatic BCAA breakdown. Therefore, BCAA catabolic control in response to insulin will be interrogated in AD mice. Hyperinsulinemic euglycemic clamps will be performed based on the inventors prior findings.

### Example 2. Test if Manipulating Dietary BCAA Intake Alters AD Progression

Cognitive functions and Ap deposits and pTau protein in AD mice can be examined following dietary BCAA restriction or supplementation for months. The genes involved in neuronal/cellular functions in the hippocampus and neocortex will be assessed for potential mechanisms.

Branched-chain amino acids and the CNS; BCAAs (i.e., leucine, isoleucine, and valine) are essential amino are not produced in our body, hence the need to obtain them from food. While BCAA acids that supplementation is widely used for improving physical fitness³⁷⁻³⁸ and for preventing muscle wasting in patients⁴⁰⁻⁴⁹ with cirrhosis, kidney failure, cancer, or sepsis mounting evidence suggests that BCAAs or their derived metabolites can induce neural oxidative stress^{4,5,8,9}, mitochondrial dysfunction^{50,51} and apoptosis^{6,7}. In support of these findings, individuals with maple syrup urine disease, a rare genetic disease that causes a mutation in genes critical BCAA-degrading enzyme complex, have nearly 10-fold higher BCAAs and their toxic keto-acids in the plasma compared to healthy controls^{52,53} leading to serious neurological impairments.

Further evidence correlating BCAAs to brain function comes from the observation that the enzyme branched-chain aminotransferase (BCAT) converts BCAAs to glutamate, an excitatory neurotransmitter. Moreover, BCAAs compete for the same large amino acid transporter (LAT) with aromatic amino acids like phenylalanine, tyrosine, and tryptophan—the precursors of neurotransmitters like dopamine, norepinephrine, and serotonin. Thus, excess circulating BCAAs may lead production of too much glutamate that contribute excitotoxicity, as well as trigger a significant imbalance and reduction some key neurotransmitters in the brain.

The inventor recognized that such metabolic diseases share a number of abnormal features with AD. These findings further underscore that BCAA control is similarly impaired in AD, however prior to the present invention the role of BCAAs and their metabolism in the onset of AD, or the CNS health in general was unknown.

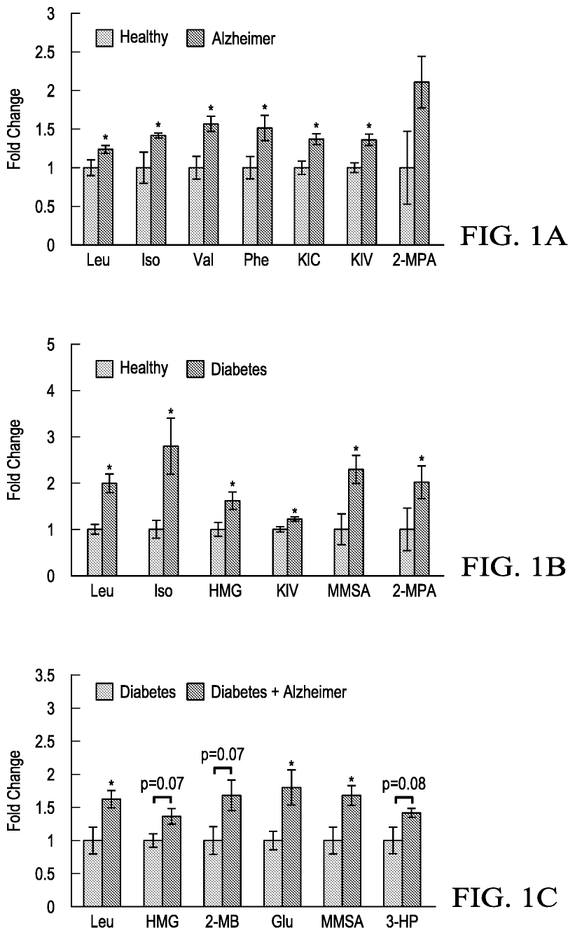

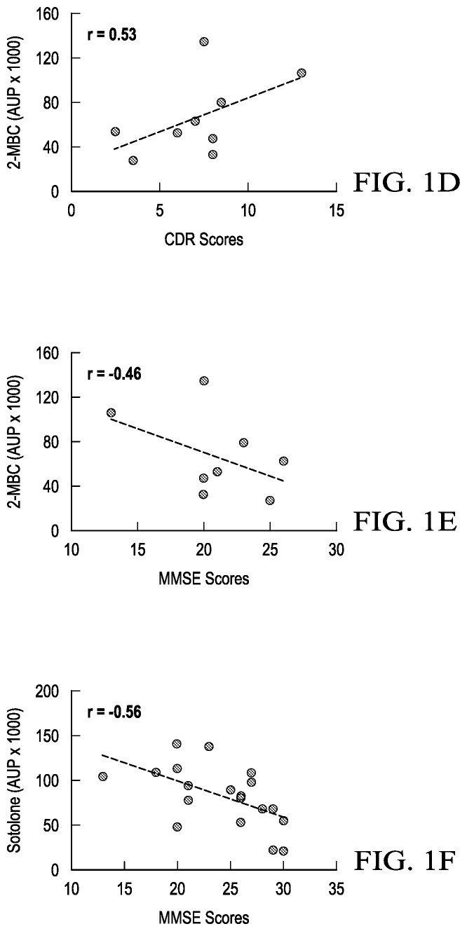

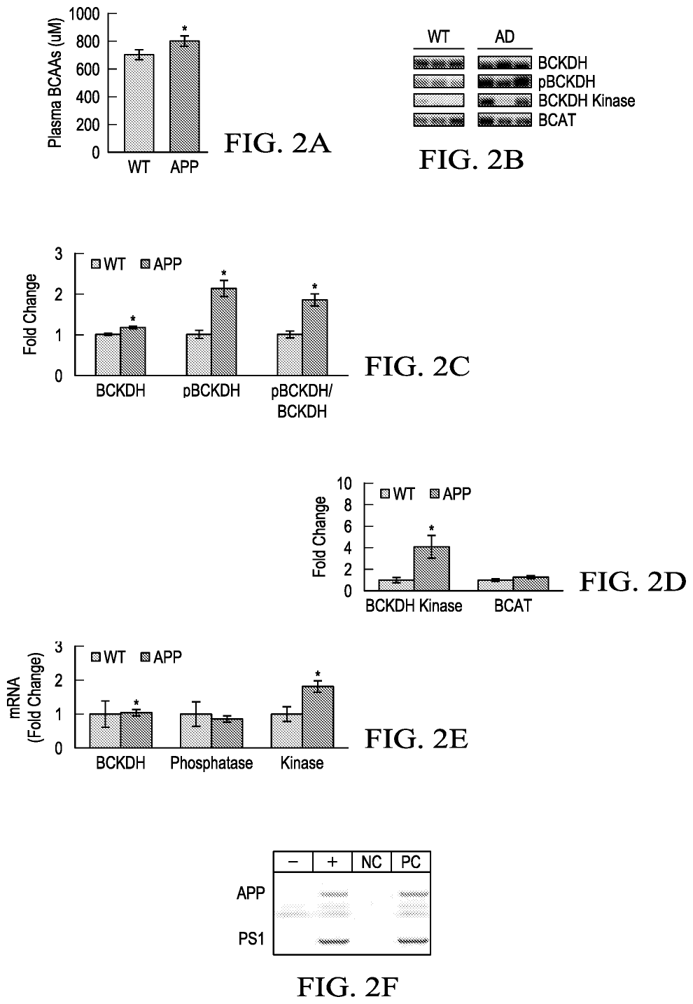

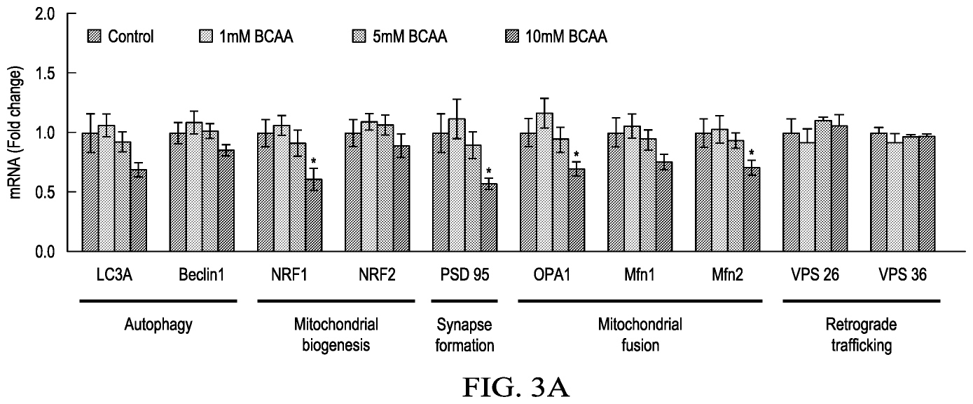

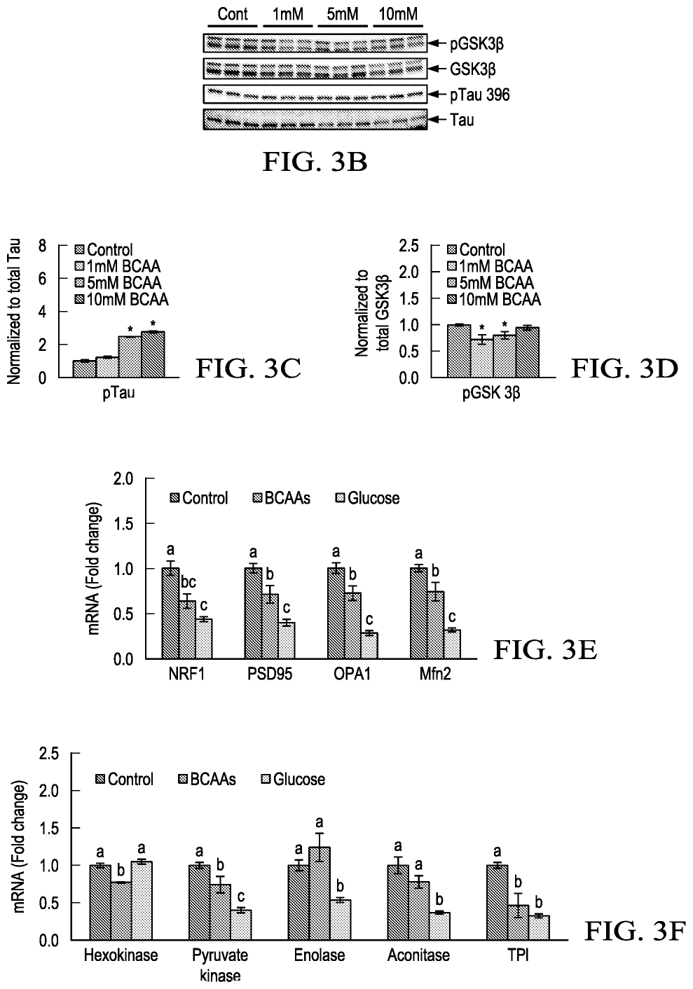

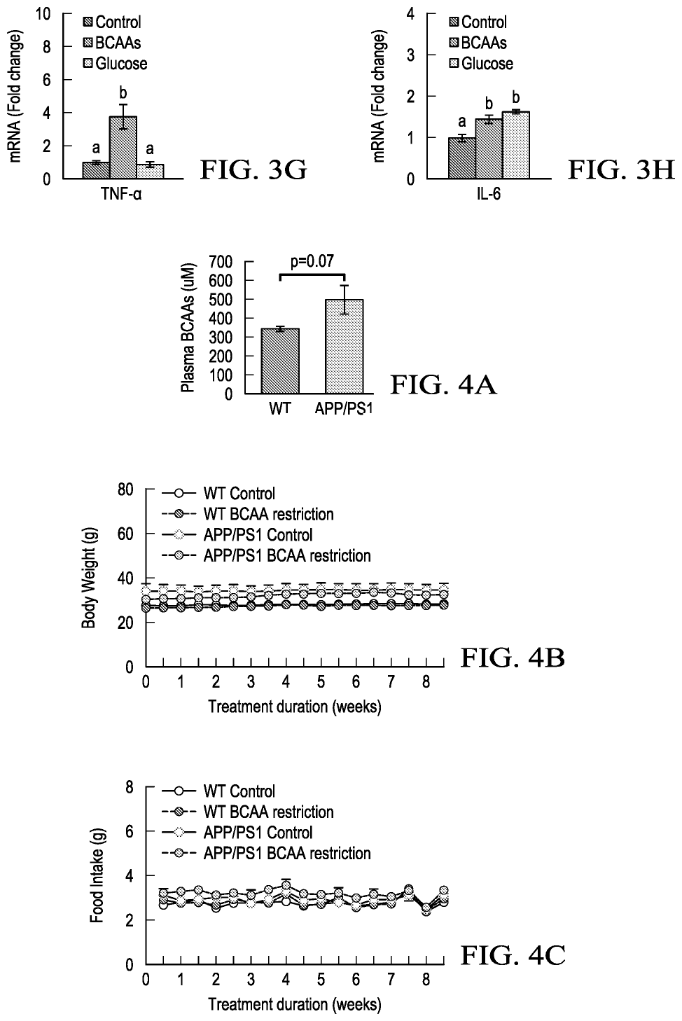

Recent clinical studies have tried to address this and examine the association between BCAAs and AD, but the findings are inconsistent⁵⁰⁻⁶². Thus, the present invention looked at fasting plasma BCAAs and/or their metabolites are increased in AD patients (FIG. 1A) as well as in a transgenic mouse model of AD (FIG. 2). Further, BCAAs were found to be a significant composite predictor of AD. More importantly, patients with AD+T2D have even higher plasma BCAAs and/or their metabolites than patients with T2D alone (FIG. 1C), indicating a unique relationship between BCAAs and AD beyond the effect of diabetes. These observations that BCAAs their derived metabolites serve novel AD biomarker that can complement other existing biomarkers to increase the efficacy of early screening and detection. Thus, a simple blood test for BCAA levels can be used to continuous monitoring of AD, and the progression and evaluating therapeutic interventions. In addition, it was found (FIGS. 3A to 3H) that BCAA supplementation in hippocampal neurons gives features of neuronal dysfunction similar to those commonly observed in AD. Thus, beyond serving as a biomarker of AD, BCAAs play a causative role in AD pathogenesis.

The present invention provides significant and relevant information related to human health because it enabled the determination of circulating BCAAs and/or their metabolites as a predictive/diagnostic biomarker for AD. Individuals may be easily tested for their susceptibility to or the onset/progression of AD by measuring blood levels of BCAAs and their metabolites in conjunction with existing markers, with minimal risks and invasiveness. In addition, the study will provide a novel dietary interventional strategy that can possibly delay further progression of AD symptoms, which evidently has significant implications in the financial burden and the quality of life for AD patients and their families.

Previously unidentified role of BCAAs in the CNS function neurodegenerative diseases such as AD in vivo. The present invention provides a novel biomarker that can be used to either predict or diagnose AD. The detection of circulating biomarkers is ideal compared to neuroimaging techniques or CSF analysis because it is simpler, less invasive, cost-effective, and patient-friendly. As a novel biomarker, BCAAs significantly improve the clinical utility and predictive power of existing methods of AD assessment.

Test the efficacy of dietary BCAA intake on AD progression using iso-caloric and isonitrogenous diet. A multi-dimensional approach (transgenic, integrative physiology, molecular biology, and behavioral) is used to understand the impact of BCAAs and their metabolism in the development of AD.

Few earlier rodent studies have demonstrated sex differences in diurnal regulation of BCAA metabolism^{53,84}. The inventor has shown that hepatic BCAA-degrading enzyme is lower in obese and diabetic men compared to lean men, but the difference disappears in women, suggesting potential sex/hormonal differences in the control of BCAA metabolism in humans well. Thus, studying both sexes is deemed necessary.

### Example 3. Examine BCAA Metabolism in a Mouse Model of Alzheimer's Disease

Examining the levels of BCAAs in both sexes is important because, in addition to its catabolic capacity, circulating amino acids (AA) like BCAAs closely reflect immediate dietary BCAA intake. This is due to the lack of the body's AA-storing mechanism for later use, unlike in the case of excess carbohydrate or fat. It is shown herein that: (1) elevated plasma BCAAs and their metabolites in both AD men and mice, (2) even higher levels in patients with AD+T2D vs. T2D alone, and (3) BCAAs as a significant composite predictor AD (FIGS. 1A-1D and 2A-2F; after 2; overnight fasting to eliminate nutritional status as a confounding variable). Along with previous finding of the inventor, higher plasma BCAAs in AD mice even before displaying any AD-like symptoms, it is shown here that plasma BCAAs and their metabolites serve as either a predictive and/or diagnostic marker of AD.

Therefore, will conduct a time-course study using double-transgenic APP/PS1 AD mice^{69,71} (FIG. 2F validation of APP/PS1 line in the inventors' breeding colony) to examine BCAA metabolism before and after development of AD-like symptoms. Unlike in humans, natural aging does not lead to AD in rodents, hence requiring other means to artificially generate AD-related brain pathology and cognitive deficits in mice. APP/PS1 transgenic mouse is a well-established AD model, studies that shed light on many important cellular and molecular mechanisms related to brain dysfunctions in AD.

### Example 4. Test if Plasma and Brain BCAA Profiles are Associated with AD

A transgenic APP/PS1 mouse model of AD is used because exhibit cognitive impairment at the age of 7-8 months. Thus, two month-old WT littermates and APP/PS1 (AD) mice can be divided into two cohorts with one cohort subjected to cognitive behavioral tests when they are 3 months old (intact cognition), and the other when they are 8 months old (4 groups total for each sex; n=13/group; 3 month-old WT or APP/PS1 mice; 8 month-old WT or APP/PS1 mice). These animals are readily available since they were breed the transgenic mice at the Texas Tech animal facility. Animals throughout the proposal will be placed on ad libitum chow diet and water unless otherwise noted. They will be group-housed and their weekly body weight and food intake will be monitored. To assess cognitive function at both 3 and months of age, the mice will undergo widely used behavioral tests: Y-maze with spontaneous alternation and Morris Water Maze. Y-maze is often used to assess working memory in animals. This method takes the advantage of rodent's tendency to explore a new arm of the maze rather than a familiar arm, and this executive action requires multiple brain regions like basal forebrain, hippocampus, and prefrontal cortex. Morris Water Maze (MWM) is another widely used behavioral test to study spatial learning and memory in rodents. Details of these behavioral assays are described by the inventor.⁷⁵ One week of rest will be given between each behavior test. Four days after the last behavioral test trial, overnight-fasted animals can be sacrificed and blood through cardiac puncture can be collected for plasma isolation. Peripheral tissues like liver, muscle, and white adipose tissue (WAT) will be harvested, snap frozen, and stored at −80° C. Brains will be also harvested and the neocortex and hippocampus will be dissected and immediately frozen. The P1 is highly experienced in rodent neuroanatomy and microdissection^{19,75,78}. Total plasma, cortical, and hippocampal BCAAs will be measured by spectrophotometric assay that measures NADH generated from BCAA oxidation's. Individual BCAAs, their derived keto acids and acylcarnitines can be analyzed by LC-MS. From the neocortex and hippocampus, branched-chain aminotransferase (BCATc)—the first enzyme in the BCAA degradation pathway found in neurons, branched-chain a-keto acid dehydrogenase (BCKDH) the rate-limiting enzyme, BCKDH phosphatase and kinase-BCKDH activating and inhibiting enzyme, respectively, the phosphorylated (inactive) state of the enzyme BCKDH/BCKDH (inactivation index)^{19,80} and KIV oxidation (i.e., functional BCKDH activity readout)¹⁹ will be determined altogether in order to comprehensively assess BCAA catabolism. The cortical and hippocampal aggregates of insoluble AB (1-40; 1-42), soluble Ap oligomers, and phosphorylated Tau (pTau) the protein deposits thought to be responsible for cognitive deficits seen in AD patients, will be measured by ELISA and/or western blots. Thus, a correlation between BCAA metabolism to AD-related brain pathology and memory deficits is obtained as a function of time.

It can be expected that plasma BCAAs and their metabolites will be higher in 8 month-old AD mice compared to WT mice, which will be associated with their impaired cognitive/memory functions as serve assessed by the behavioral tests, indicating that plasma BCAAs and their metabolites may surrogate marker for AD-like symptoms. In the neocortex and hippocampus of AD mice, increased Af aggregates and pTau can be observed, as well as elevated BCAAs/their metabolites that are associated with impaired BCAA catabolism.

High plasma BCAAs and their metabolites in AD mice at months of age, when there is no visible AD-related cognitive dysfunction can be determined to indicate a predictive role in the onset of AD, in keeping with the inventor's findings in 5 month-old AD mice. If these changes are absent in female mice, then a role of estrogen in plasma BCAAs in AD is determined by performing an ovariectomy. However, women usually develop AD at 65 years of age or greater, at which they are well within the postmenopausal, low-estrogen stage, so estrogen may not critical mediator. Interestingly, during postmenopause there compensatory surge of follicular-stimulating hormone (FSH) from the anterior pituitary in circulation, and, thus, may have an important role other than stimulating ovarian functions. The role of FSH in the AD female mice is tested using FSH-specific antibody. While APP/PS1 mouse model was chosen here mainly because of the strong and persistent transgene expressions in the brain unlike other models, in which they are temporally induced by doxycycline (e.g., AAV-mediated transfection, to extend these findings to other AD mouse models for reproducibility).

### Example 5. Test if High Plasma BCAAs in AD are Due to Impaired Hepatic BCAA Catabolism

The findings above indicate defective BCAA degradation in liver, an organ with high BCAA catabolic activity from transgenic AD mice at both protein and gene levels (FIGS. 2B-E). This likely explains high plasma BCAAs in AD mice compared to controls (FIG. 2A). In fact, the inventor has previously shown that plasma BCAAs are primarily determined by hepatic BCAA catabolism, and that this is positively regulated by insulin, a master regulator of metabolism, as evidenced during hyperinsulinemic euglycemic clamps in rodents. Thus, to mechanistically test if BCAA catabolic control is indeed impaired in AD mice, it is necessary to assess hepatic BCAA breakdown in response to insulin.

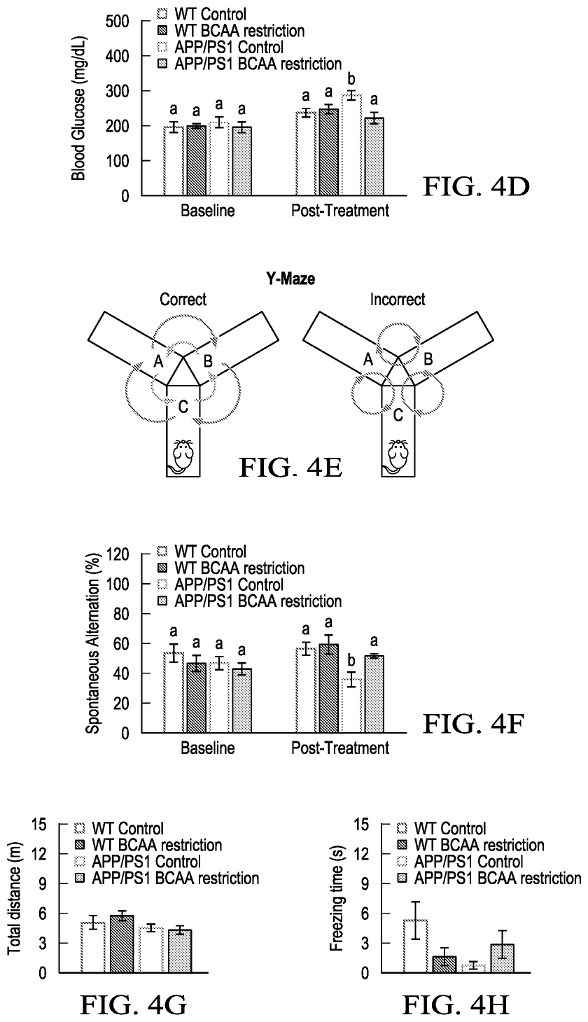

Design and Analysis: Observe sex differences studying females and males. 8 month-old WT or transgenic AD (APP/PS1) mice (4 groups total for each sex; n=13/group; WT or APP/PS1 mice+ or 4mU clamps) will be implanted with jugular and arterial catheters. After recovery (usually 4-5 days), the animals will undergo hyperinsulinemic euglycemic clamps (FIGS. 4A-D). The purpose hyperinsulinemic clamps is to minimize potential confounding variables such performing changes in plasma glucose or insulin that may arise from insulin injection/infusion. By maintaining fixed insulin levels and euglycemia, clamp studies allow to eliminate potential effects of glucose and isolate the role of insulin action in the control of BCAA metabolism. Selected high insulin dose (4mU) was shown to be effective at suppressing plasma BCAAs in WT mice (FIGS. 4A-D), hence validating the efficacy of the dose. Tracer dilution technique will be incorporated in clamps to assess glucose flux to verify insulin action, which is defined the ability of insulin to suppress hepatic glucose production (hGP). After 5 hours of fasting, mice will receive primed continuous intravenous infusion of [U-¹³C-Glucose (0.6 umol/kg/min) at t=−100 min. Clamps will start with a primed continuous infusion of insulin at t=0 and last for 120 min. Somatostatin will be co-infused to inhibit endogenous insulin and glucagon secretion from pancreas and counter-regulatory hormones such as corticosterone and growth hormone, as well as gastrointestinal hormones that may affect nutrient metabolism. Blood glucose will be assessed every 10 minutes during the clamps to allow for adjustment o glucose infusion rates to maintain stable basal glucose. Blood samples will be collected in EDTA tubes through an arterial catheter at multiple pre- and post-clamp time-points for glucose flux. Minimize blood loss, erythrocytes will be mixed with saline and infused back. Plasma insulin will be measured by ELISA to confirm its rise during hyperinsulinemic clamps. Animals can be sacrificed at the end of the clamps and tissues and blood samples will be processed as described above. Importantly, abundance of the enzymes responsible for BCAA degradation and their activity in liver and other peripheral tissues will be assessed.

WT mice will have lower plasma BCAAs and their derived metabolites during hyperinsulinemic clamps compared to 1 mU basal clamps. WT mice will also display enhanced hepatic BCAA catabolism increased BCKDH protein and activity during hyperinsulinemic clamps. On other hand, the ability insulin to increase hepatic BCAA degradation will be diminished in APP/PS1 mice, resulting in higher plasma BCAAs and their metabolites. These outcomes would show that APP/PS1 mice indeed have impaired hepatic BCAA metabolism. BCAA catabolism in other peripheral insulin-sensitive tissues like white adipose tissue and muscle may be impaired as well in APP/PS1 mice, however, this may be unlikely as these organs play a minor role regulating BCAAs. Protein turnover can be assessed to test if protein catabolism is a major contributor of elevated plasma BCAAs in AD mice.

### Example 6. Manipulating Dietary BCAA Intake Alters AD Progression

Dietary intervention has been shown to lower the susceptibility to age-related cognitive impairment or AD. A nutritional strategy aimed at decreasing AD pathology is used. Others have shown that protein restriction improves cognitive performance in AD mice⁸⁸ although the role of BCAAs in this limited protein intake is unknown. A body of literature points to the deleterious effects of BCAA overload on neuronal functions and neurotransmitter balance in the brain149.

### Example 7. Serum BCAAs and their Metabolites are Associated with AD

To first determine a possible link between circulating BCAA levels and AD, serum samples from male individuals with AD or age-matched healthy individuals were analyzed through untargeted metabolomics by LC-MS. From 95 metabolites that are found to be differentially regulated, Compound Discoverer database identified BCAA metabolism as one of the most affected biological pathways in AD patients. As shown in FIG. 1A, all BCAAs including leucine, isoleucine, and valine as well as their derivatives such as keto-isocaproate (KIC; metabolite of leucine) and keto-isovalerate (KIV; metabolite of valine) were significantly higher in AD group compared to those in healthy controls, with an increase ranging from 20-50% (FIG. 1A). Importantly, other amino acids in the serum with the exception of phenylalanine were not different between healthy and AD individuals. T2D is known to share a number of abnormal features with AD, and it is now widely accepted that systemic BCAA levels are elevated in insulin resistant or diabetic state. To test if having both conditions magnify BCAA-raising effects, samples from patients with T2D or T2D+AD were also obtained and subjected to metabolomics platform. Serum BCAAs (100-180%) and their oxidized intermediates including 3-methylglutarylcarnitine (HMG; metabolite of leucine; 60%) and methylmelonate semialdehyde (MMSA; metabolite of valine; 120%) were significantly elevated in T2D group compared to those in healthy control group as expected (FIG. 1). Interestingly, individuals with both T2D and AD displayed even higher levels of leucine (60%) and its metabolite HMG (35%), glutamate (80%), and oxidized metabolites of valine such as 2-methylbutyryl (2-MB; 65%), MMSA (60%), and 3-hydroxypropanoate (3-HP; 40%) compared to individuals with T2D alone (FIG. 1C), suggesting an association between BCAAs and AD extending beyond the effect of T2D. To examine this relationship further, Clinical Dementia Rating (CDR) and Mini-Mental State Examination (MMSE) scores were obtained from the same healthy and AD individuals, and conducted a correlation analysis between the scores and the serum levels of BCAA-related metabolites. The sum of CDR scores ranges from 0 (normal) to 18 (severe dementia), and in MMSE a score between 25-30 points to normal cognition while a score less than 12 indicates severe dementia. The inventors observed that 2-methylbutyrylcarnitine (2-MBC), a metabolite of isoleucine, is strongly associated with both CDR scores (r=0.53; FIG. 1D) and MMSE scores (r=−0.46; FIG. 1E). Another metabolite of isoleucine, sotolone, was strongly and inversely correlated with MMSE scores (r=−0.56;

FIG. 1F). Collectively, these findings strongly suggest that circulating BCAAs and/or their metabolites are positively associated with AD progression in humans.

### Example 8. BCAA Catabolism is Impaired in APP_{swe} Transgenic Mice

Transgenic AD mice reveal (share) remarkable similarities with humans in regards to the formation of amyloid plaques, neurofibrillary tangles, and related cognitive decline, thus serving as an excellent model to study the disease pathophysiology³⁴. APP_{swe} mouse (also known as Tg2576) is one of the most widely used mouse models of AD that overexpresses human amyloid precursor protein (APP) containing Swedish mutation and shows a cognitive impairment as early as 6 months of age³⁵. The inventors measured plasma BCAA levels from 8-month-old APP_{swe} mice to determine if a relationship similar to what the inventors observed in humans exists in these mice. Indeed, plasma BCAAs were significantly elevated in APP_{swe} mice compared to WT controls (FIG. 2A), establishing a basis to use transgenic AD mice such as this model to further investigate the disease progression and its underlying mechanisms. The inventors and others have previously shown that circulating BCAA levels are regulated primarily by hepatic BCAA catabolism. Branched-chain a-ketoacid dehydrogenase (BCKDH) is the rate-limiting enzyme in BCAA breakdown that becomes inactive when phosphorylated by BCKDH kinase. Protein analysis from liver demonstrated significantly higher pBCKDH in APP_{swe} mice compared to that in WTs, and so was the inactivity index as expressed by pBCKDH/BCKDH ratio (FIGS. 2B, C). In support of these results, APP_{swe} mice also showed higher protein expression of BCKDH kinase without affecting the first enzyme in BCAA degradation pathway, branched-chain aminotransferase (BCAT; FIG. 1D). This is further confirmed by a markedly increased mRNA of hepatic BCKDH kinase in APP_{swe} mice (FIG. 1E). Altogether, these data suggest that the association between high circulating BCAA levels and AD extends to a transgenic mouse model such as APP_{swe}, and that this may be primarily due to impaired hepatic BCAA catabolism in AD.

### Example 9. BCAA Supplementation Induces AD-Like Changes and Disrupts Cellular Functions in HT-22 Neurons

Elevated systemic levels of BCAAs and their metabolites in AD raise a possibility that BCAAs and AD are causally linked. To test this, 0, 1, 5, or 10 mM of a mixture of BCAAs (leucine, isoleucine, valine; 1:2:1 ratio) was supplemented to differentiated HT-22 hippocampal neurons for 24 h. The in vitro results showed that BCAA exposure dose-dependently downregulates mRNA of neuronal health markers such as LC3A (autophagy), NRF1 (mitochondrial biogenesis), PSD 95 (synapse formation), as well as OPA1, Mfn1, and Mfn2 (mitochondrial fusion; FIG. 3A). It is important to note that these genes are among the ones commonly found to be impaired in AD brain, and these impaired neuronal functions in AD are clearly demonstrated by increased oxidative stress and a dramatically reduced synaptogenesis and ATP production. Moreover, the inventors observed a significant increase in pTau in BCAA-treated HT-22 neurons compared to that in vehicle-treated neurons (FIGS. 3B, C), and this is most likely attributed to a significant decrease in phosphorylated, inactive state of GSK3β (FIG. 3D), an enzyme responsible for phosphorylating Tau protein that leads to formation of paired helical filaments (PHF) and neurofibrillary tangles (NFT). Neurons have a high demand for glucose for important functions including the synthesis of ATP and neurotransmitters³⁸, thus making proper glucose metabolism crucial for the overall health and communication between neurons. To further understand the detrimental effects of BCAA exposure on neuronal function and establish a mechanistic link to AD, the inventors treated differentiated HT-22 neurons with BCAAs and examined the glycolysis pathway. A significant downregulation of genes responsible for mitochondrial biogenesis and fusion as well as synapse formation was verified in an independent cohort (FIG. 3E). As expected, BCAA supplementation for 24 h was enough to dramatically lower mRNAs that encode enzymes involved in glycolysis, including the rate-limiting enzymes hexokinase and pyruvate kinase (FIG. 3F). Interestingly, the harmful effects were comparable to those from a high-glucose treatment, a widely used in vitro neurotoxicity model for establishing neuronal stress, disrupted cellular metabolism, and apoptosis. In agreement with BCAA-induced neuronal dysfunction, mRNA abundance of pro-inflammatory mediators TNF-α and IL-6 was markedly increased compared to vehicle-treated HT-22 neurons (FIGS. 3G, H). These findings show that BCAAs cause multiple neuronal dysfunctions as commonly observed in AD at least partly by impairing cellular glycolytic and bioenergetic pathways in neurons.

### Example 10. BCAA-Restriction Diet Delays Onset of Cognitive Decline in APP/PS1 Mice

Given the strong association between circulating BCAAs/metabolites and AD (FIGS. 1, 2), and the dose-dependent downregulation of neuronal health markers by BCAA supplementation (FIG. 3), it seems likely that BCAAs are causally linked to AD development. Rather than providing more BCAAs, the inventors determined that limiting BCAA consumption would be a more physiological approach to test the hypothesis. To this end, WTs and APP/PS 1 transgenic mice were placed on either a regular chow diet or a customized diet that is deficient of individual BCAAs by 50% for two months. The special diet was formulated to restrict the amount of all three BCAAs while being isocaloric and isonitrogenous compared to the regular chow diet (Table 1).

TABLE 1
isocaloric and isonitrogenous compared to the regular chow diet.

| | Control Diet | | (50%) BCAA-restricted Diet | |
|---|---|---|---|---|
| | Research Diets | | Research Diets | |
| | A11072001 | | A20112501 | |
| | Gm | Kcal | Gm | kcal |
| Macronutrients | | | | |
| Protein | 34 | 33 | 34 | 33 |
| Carbohydrate | 49 | 49 | 49 | 49 |
| Fat | 8 | 18 | 8 | 18 |
| Fiber | 4 | 0 | 4 | 0 |
| Total | 91 | 100 | 91 | 100 |
| Kcal/gm | 4 | | 4 | |
| Amino Acids | | | | |
| Cystine | 5.5 | 22 | 6.29 | 25 |
| Isoleucine | 17.88 | 72 | 8.94 | 36 |
| Leucine | 31.63 | 127 | 15.81 | 63 |
| Lysine | 24.75 | 99 | 29.28 | 117 |
| Methionine | 6.88 | 28 | 7.86 | 31 |
| Phenylalanine | 17.88 | 72 | 17.88 | 72 |
| Threonine | 15.13 | 61 | 17.29 | 69 |
| Tryptophan | 5.5 | 22 | 5.50 | 22 |
| Valine | 23.38 | 94 | 11.69 | 47 |
| Histidine | 8.25 | 33 | 9.43 | 38 |
| Alanine | 12.38 | 50 | 14.15 | 57 |
| Arginine | 27.5 | 110 | 32.08 | 128 |
| Aspartic Acid | 20.63 | 83 | 23.58 | 94 |
| Glutamic Acid | 37.13 | 149 | 43.43 | 174 |
| Glycine | 38.5 | 154 | 45.00 | 180 |
| Proline | 17.88 | 72 | 20.43 | 82 |
| Serine | 15.13 | 61 | 17.29 | 69 |
| Tyrosine | 12.38 | 50 | 12.38 | 50 |
| Other Ingredients | | | | |
| Corn Starch | 386 | 1544 | 386 | 1544 |
| Maltodextrin | 100 | 400 | 100 | 400 |
| Cellulose, BW200 | 40 | 0 | 40 | 0 |
| Soybean Oil | 25 | 225 | 25 | 2255 |
| Lard | 55 | 495 | 55 | 495 |
| Mineral Mix S10026 | 10 | 0 | 10 | 0 |
| Dicalcium Phosphate | 13 | 0 | 13 | 0 |
| Calcium Carbonate | 5.5 | 0 | 6 | 0 |
| Potassium Citrate, 1H20 | 16.5 | 0 | 17 | 0 |
| Sodium Bicarbonate | 3.5 | 0 | 3.5 | 0 |
| Vitamin Mix V10001 | 10 | 40 | 10 | 40 |
| Choline Bitartrate | 2 | 0 | 2 | 0 |
| Cholesterol | 0782 | 0 | 0.782 | 0 |
| Total | 1005.64 | 4057 | 1005.592 | 4057 |

The inventors predicted that if BCAAs are indeed a significant contributor to AD pathogenesis, then lowering BCAA intake would at least partly slow down the disease progression. APP/PS1 mice displayed higher plasma BCAA levels compared to WTs before dietary treatment (FIG. 4A), thus confirming the inventors' earlier data from APP_{swe} mouse model. Diet change did not affect body weight or food intake for the entire treatment duration (FIGS. 41B, C). Metabolic dysregulation such as insulin resistance or impaired glycemic control is often associated with AD^{42,43}. While there was no difference in blood glucose between groups at baseline, after two months the inventors observed a significantly increased blood glucose in regular chow-fed APP/PS1 mice compared to that in WT controls, as well as their own baseline (FIG. 4D). Interestingly, BCAA restriction was able to prevent the rise of blood glucose in APP/PS1 mice (FIG. 4D). Next, the inventors determined the effects of limiting BCAA intake on the cognitive function in these mice. Y-maze (FIG. 4E) is a widely used behavioral test used to assess working memory. APP/PS1 mice did not show any differences in spontaneous alternation compared to WTs at baseline, most likely because they were not old enough to show any cognitive dysfunction (ex. spatial learning) which usually occurs between 12-13 month of age (FIG. 4E). As expected, they displayed poor memory after two months on chow diet as evidenced by lower spontaneous alternation, but APP/PS1 mice on BCAA-restriction diet resisted the decline and they were not different from WT mice on either diet (FIG. 4F). To rule out the possibility that the behavioral outcome induced by BCAA restriction is partly due to changes in the mobility or distraction of the animals, the inventors carefully examined the total distance (FIG. 4G), freezing time (FIG. 4H), number of entries (FIG. 4I), and mean speed (FIG. 4J), and none of the parameters were found to be markedly different between groups. These results suggest that restricting BCAA consumption substantially delays AD progression independent of changes in energy balance, and this cognitive benefit is associated with improved glycemic control.

### Example 11. BCAA-Restriction Diet Lowers AD-Related Pathology and Restores Neurotransmitter Levels in the Cortex and Hippocampus in APP/PS1 Mice

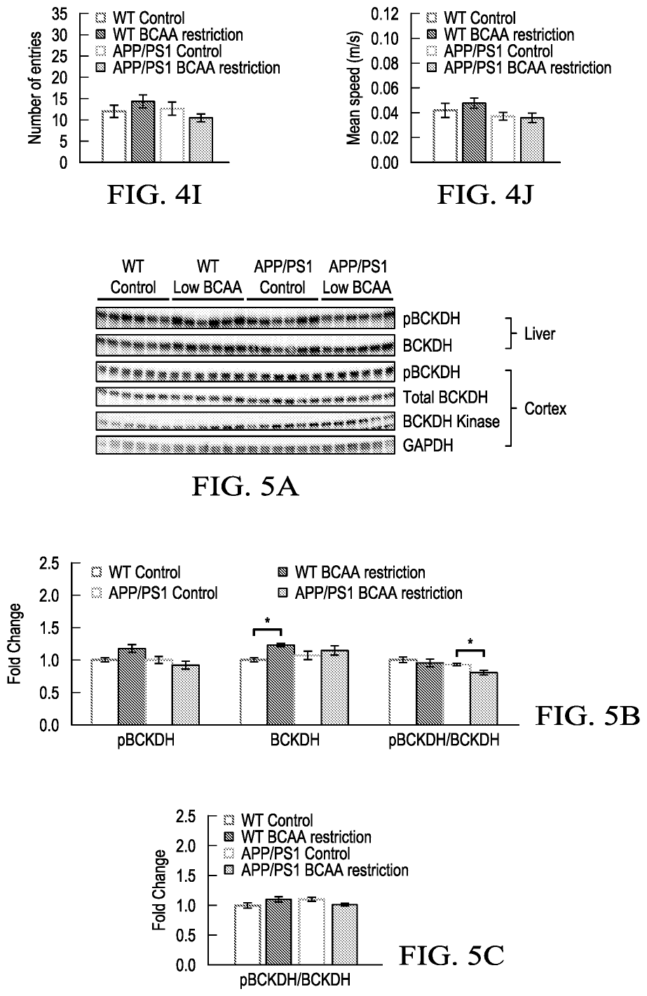

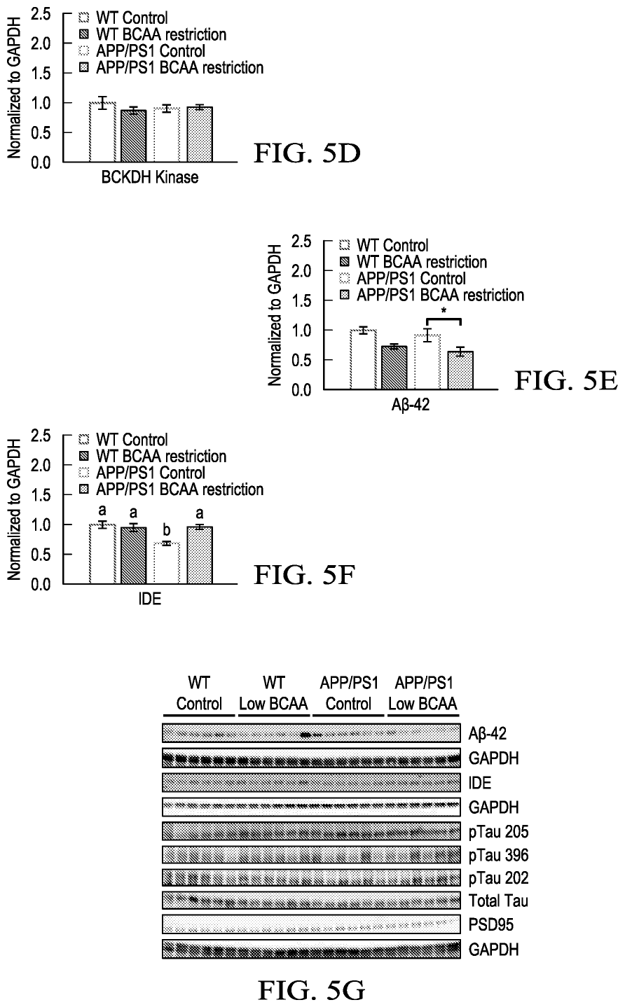

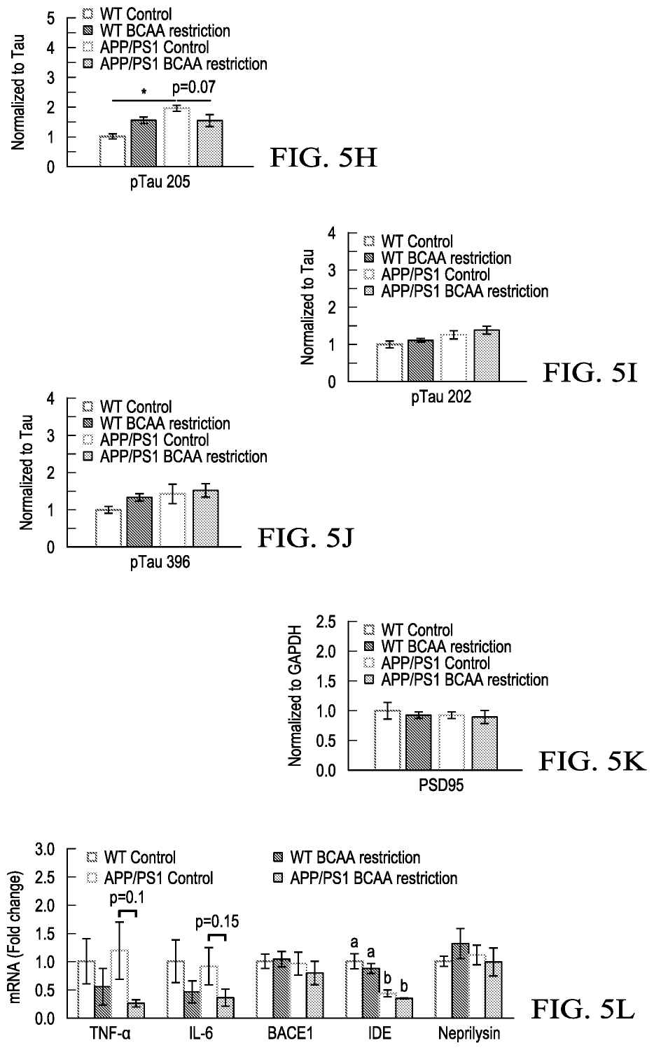

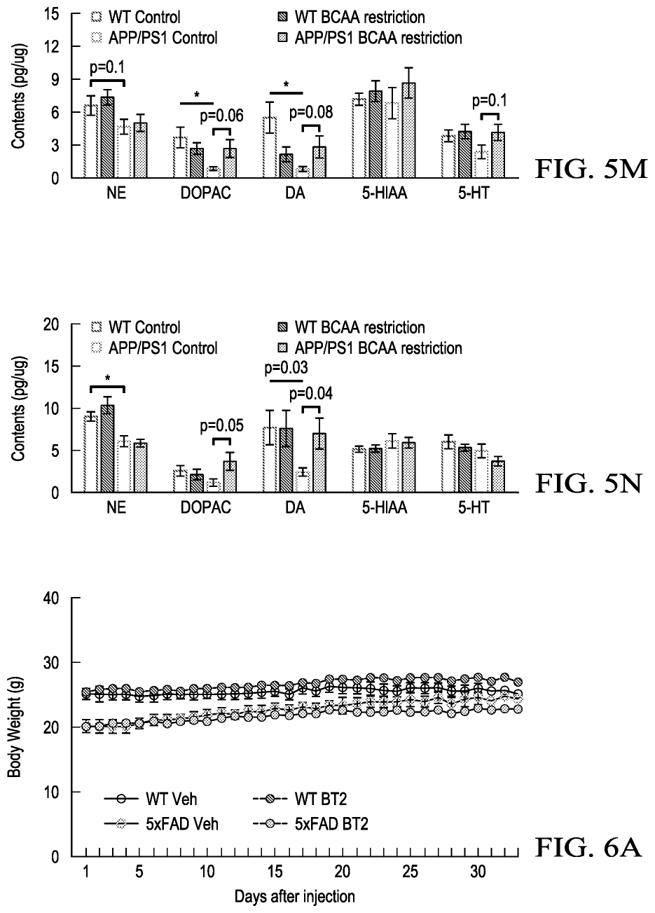

To understand possible mechanisms by which limiting BCAA intake slows down AD development, the inventors focused on examining key markers of AD-related brain pathology and neurotransmitter levels in the hippocampus and cortex as an indicator of neuronal damage and health. BCAA-restricted APP/PS1 mice had a significantly lower ratio of pBCKDH to BCKDH in liver compared to regular chow-fed APP/PS1 mice (FIGS. 5A and 5B), indicating enhanced hepatic BCAA catabolism that would likely lead to lower circulating BCAAs. However, the inactivity index and BCKDH kinase protein expression were not different in the cortex across groups (FIGS. 5C, D). Next, the inventors assessed amyloid peptide and phosphorylated Tau levels in the cortex as these are considered to be the primary culprits driving AD progression. APP/PS1 mice fed BCAA-restricted diet displayed significantly lower Aβ-42 levels compared to the group on a regular chow diet (FIG. 5E). In keeping with this finding, while APP/PS1 controls showed a markedly decreased protein expression of insulin-degrading enzyme (IDE), the enzyme known to break down amyloid peptide, BCAA restriction completely reversed it (FIG. 5F). Furthermore, limiting BCAA intake was able to nullify the increased, phosphorylated state of Tau at Thr205, but not at Ser202 or 396, in APP/PS1 mice compared to those fed a regular chow diet (FIGS. 5G-J). Contrary to what was expected based on the effects of BCAA supplementation on HT-22 hippocampal neurons, PSD95 protein was not increased in BCAA-restricted APP/PS1 mice compared to chow-fed counterparts or WTs (FIG. 5K). Neuroinflammation induced by microglia is thought to be primarily triggered by soluble Ab oligomers and play a central role in promoting formation of Tau pathology. In line with lower Aβ-42 and pTau levels in the cortex, APP/PS1 mice fed BCAA-restricted diet displayed a trend of decreased pro-inflammatory markers such as TNF-α and IL-6, without any changes in Aβ-degrading enzymes including IDE and neprilysin (FIG. 5L). Sufficient neurotransmitter (NT) synthesis or concentration is essential for the overall brain health, and lower monoaminergic neurotransmitters such as norepinephrine (NE), dopamine (DA), and serotonin (5-HT) are observed in AD brains². With more recent studies demonstrating that lower NT levels or degeneration of NT-synthesizing neurons are often observed in the early phase of AD development, preventing or reversing the neuronal deterioration is expected to alleviate AD progression and related symptoms. Indeed, regular chow-fed APP/PS1 mice showed reduced levels of NE, DA and its metabolite DOPAC, and 5-HT in the cortex and hippocampus compared to WTs, but BCAA restriction for two months was able to completely normalize the NT concentrations in both brain regions, most notably DA and DOPAC (FIGS. 5M, N). Altogether, these data suggest that BCAA restriction effectively lowers amyloid and Tau pathology that is associated with reduced inflammation in the hippocampus of APP/PS1 mice. This is also linked with reinstatement of monoamine neurotransmitters in both cortex and hippocampus, which explain the observed improvement in cognitive function.

### Example 12. BT2, a BCAA-Lowering Compound, Effectively Reduces Aβ-42 and Enhances Cortical and Hippocampal Neurotransmitter Levels in 5xFAD Mice

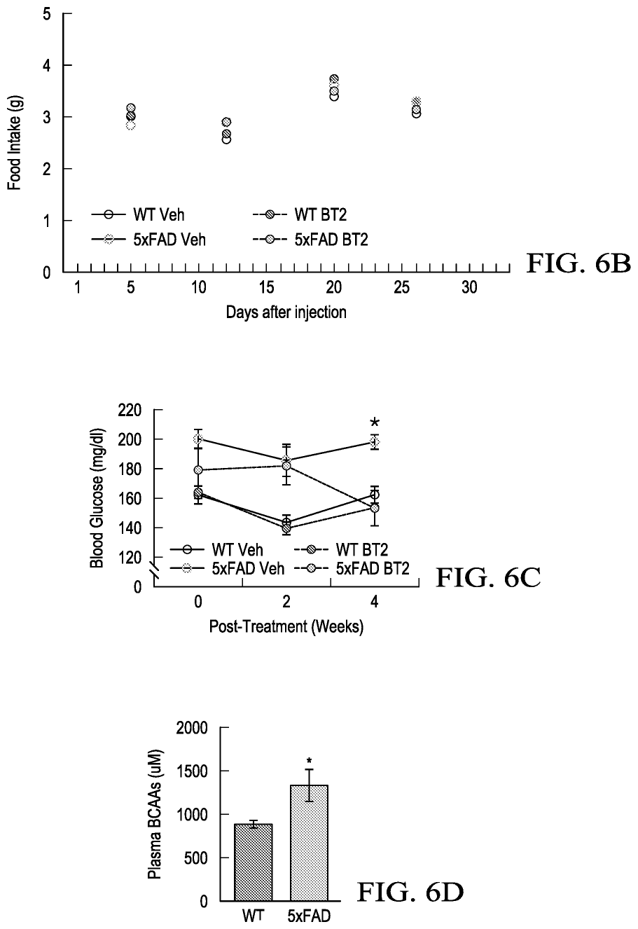

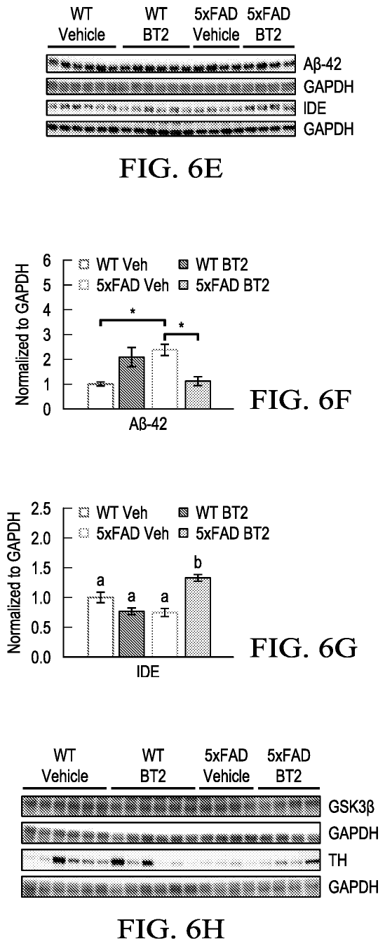

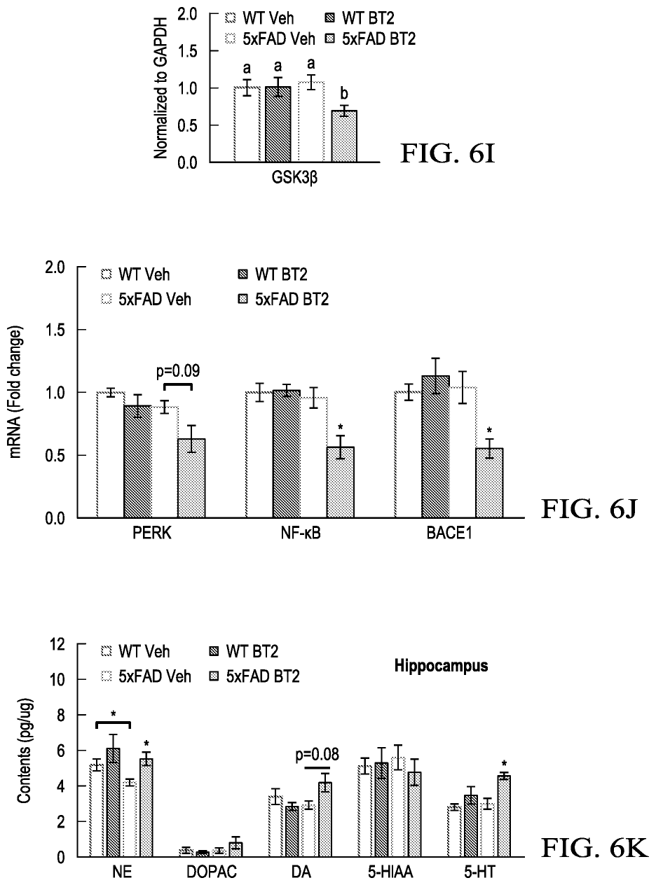

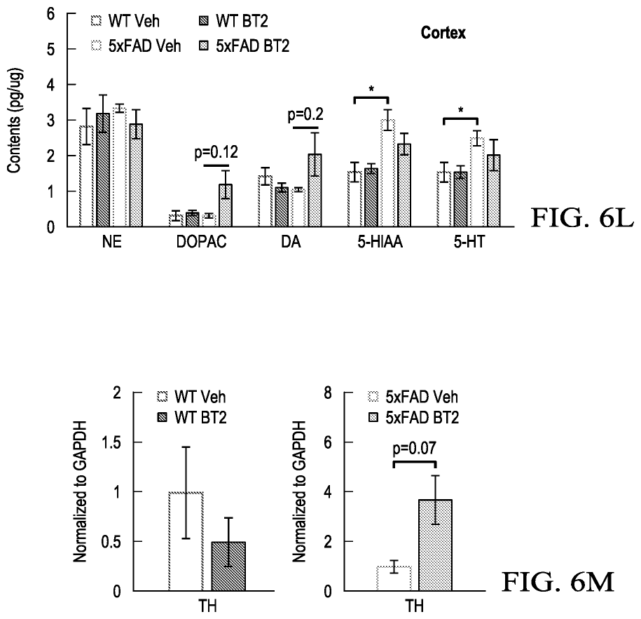

As a complementary strategy to dietary BCAA restriction, the inventors used a pharmacological approach to lower circulating BCAAs and examine the beneficial effects in AD mice. BT2 is an allosteric inhibitor of BCKDH kinase that suppresses BCKDH activity, leading to increased BCAA catabolism and lower plasma BCAA levels. For one month, BT2 (40 mg/kg ip) was daily injected to 5xFAD mice, another widely used AD model that overexpresses three APP and two presenilin (PSEN1) mutations. These transgenic mice develop amyloid plaques and neuroinflammation at as early as 1.5-2 months of age⁷, thus allowing the inventors to investigate the role of BCAAs in AD-related brain pathology when they are young. Body weight (FIG. 6A) and food intake (FIG. 6B) were not different between WT and 5xFAD mice regardless of treatments. Supporting the data from APP_{Swe} and APP/PS1 mouse experiments, 5xFAD mice showed significantly higher plasma BCAA levels compared to WT mice at baseline (FIG. 6D). They also had elevated blood glucose compared to WTs before treatment (p<0.05; FIG. 6C). However, similar to what was observed in APP/PS1 mice fed BCAA-restriction diet, BT2-treated 5xFAD mice were able to markedly decrease blood glucose compared to vehicle-treated 5xFAD mice (FIG. 6C). No change was seen within WT groups. Next, the inventors examined amyloid and Tau pathology in the cortex. Not surprisingly, 5xFAD control group had a significantly higher Aβ-42 level compared to the WT controls, but that was completely rescued in BT2-treated 5xFAD mice (FIG. 6E). This decreased amyloid burden is most likely due to elevated Aβ-degrading enzyme IDE by BT2 (70%; FIG. 6F, FIG. 6G, FIG. 6H shows expression data. In support of reduced AD-related pathology, Tau-phosphorylating and pro-inflammatory enzyme GSK3β was significantly reduced in the cortex after BT2 treatment (FIG. 6I). In the hippocampus, mRNA of key mediators of ER stress (PERK) and inflammation (NF-κB) was decreased in BT2-treated 5xFAD mice, and more interestingly, BACE1 (a.k.a. β-secretase) that is responsible for cleaving APP to promote Aβ-42 production was markedly lowered in these mice (FIG. 6J). As in BCAA restriction experiment, the inventors sought to determine the effects of BT2 on neuronal health by measuring neurotransmitter levels in the hippocampus and cortex. These data demonstrate that 5xFAD control mice have a significant reduction of NE compared to WT controls in the hippocampus, but NE level is fully restored in BT2-treated 5xFAD mice (FIG. 6K). The inventors also observed higher DA (40%; p=0.08) and 5-HT (50%; p<0.05) after BT2 treatment. Similar increase was shown for DA (180%) and its metabolite DOPAC (300%) in the cortex of BT2-treated 5xFAD mice (FIG. 6L). To confirm greater hippocampal NE levels found in these mice, the inventors assessed protein expression of tyrosine hydroxylase (TH), the rate-limiting enzyme for synthesis of catehcolamines including NE and DA. While there was no difference between WT groups, BT2 treatment increased TH level by nearly 400% in 5xFAD mice (p=0.07; FIG. 6M). Collectively, these results suggest that BT2 successfully recapitulates the pro-neuronal effects of dietary BCAA restriction by lowering AD-related brain pathology and restoring or improving key NT levels in the brain.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “of” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. Fernstrom J D. Large neutral amino acids: dietary effects on brain neurochemistry and function. Amino Acids. September 2013; 45(3):419-430.
2. Fernstrom J D. Branched-chain amino acids and brain function. J Nutr. June 2005; 135(6 Suppl):15399-1546S.
3. Pardridge W M. Kinetics of competitive inhibition neutral amino acid transport a blood-brain barrier. J Neurochem. January 1977; 28(1):103-108.
4. Bridi Braun C A, Zorzi G K, al. alpha-keto acids accumulating in maple syrup urine disease stimulate lipid peroxidation and reduce antioxidant defences in cortex from young rats. Metab Brain Dis. June 2005; 20(2):155-167.
5. Funchal C, Latini A, Jacques-Silva M C, 1. Morphological alterations and induction of oxidative stress in glial cells caused by the branched-chain alpha-keto acids accumulating in maple syrup urine disease. Neurochem Int. December 2006; 49(7):640-650.
6. Jouvet P, Kozma M, Mehmet Primary human fibroblasts from a maple syrup urine disease patient undergo apoptosis following exposure to physiological concentrations of branched chain aming acids. Ann N Y Acad Sci. 2000; 926:116-121.
7. Jouvet P, Rustin P, Taylor D L, et al. Branched chain amino acids induce apoptosis in neural cells without mitochondrial membrane depolarization or cytochrome release: implications for neurological impairment associated with maple syrup urine disease. Mol Biol Cell. May 2000; 11(5):1919-1932.
8. Lu G, Sun H, She P. et al. Protein phosphatase 2Cm is a critical regulator of branched-chain amino acid catabolism in mice and cultured cells. J Clin Invest. June 2009; 119(6):1678-1687.
9. Oyarzabal A, Martinez-Pardo M, Merinero B, et al. A novel regulatory defect in the branched-chain alpha-keto acid dehydrogenase complex due to a mutation in the PPM1K gene causes a mild variant phenotype of maple syrup urine disease. Hum Mutat. February 2013; 34(2):355-362.
10. Ono H, Pocai A, Wang Y, et al. Activation of hypothalamic S6 kinase mediates diet-induced hepatic insulin resistance in rats. J Clin Invest. August 2008; 118(8):2959-2968.
11. Lee H K, Kwon B, Lemere C A, et al. mTORC2 (Rictor) in Alzheimer's Disease and Reversal of Amyloid-beta Expression-Induced Insulin Resistance and Toxicity in Rat Primary Cortical Neurons. Alzheimers Dis. 2017; 56(3):1015-1036.
12. Jang C, Oh S F, Wada S, et al. A branched-chain amino acid metabolite drives vascular fatty acid transport and causes insulin resistance. Nat Med. April 2016; 22(4):421-426.
13. Moghei M, Tavajohi-Fini P, Beatty B, Adegoke O A. Ketoisocaproic acid, a metabolite leucine, suppresses insulin-stimulated glucose transport in skeletal muscle cells in a BCAT2-dependent manner. Am Physiol Cell Physiol. Sep. 1, 2016; 311(3):C518-527.
14. Newgard C B, An J, Bain J R, et al. A branched-chain amino acid-related metabolic signature that differentiates obese and lean humans and contributes to insulin resistance. Cell Metab. April 2009; 9(4):311-326.
15. Wang T J, Larson M G, Vasan R S, et al. Metabolite profiles and the risk of developing diabetes. Med. April 2011; 17(4):448-453.
16. Wurtz P, Soininen P, Kangas et al. Branched-chain and aromatic amino acids are predictors of insulin resistance in young adults. Diabetes Care. March 2013; 36(3):648-655.
17. Zhang F, Zhao S, Yan W, et al. Branched Chain Amino Acids Cause Liver Injury in Obese/Diabetic Mice by Promoting Adipocyte Lipolysis and Inhibiting Hepatic Autophagy. EBioMedicine. November 2016; 13:157-167.
18. Salas I H, De Strooper B. Diabetes and Alzheimer's Disease: A Link not as Simple as it Seems. Neurochem Res. Dec. 6 2018.
19. A C, Fasshauer M, Filatova N, et al. Brain insulin lowers circulating BCAA levels by inducing hepatic BCAA Shin catabolism. Cell Metab. Nov. 4 2014; 20(5):898-909.
20. Alzheimer's. Alzheimer's Disease Facts. figures, 2019. Figures. 2018; https://www.alz.org/alzheimers-dementia/facts
21. Weuve J, Hebert L E, Scherr P A, Evans D A. Deaths in the United States among persons with Alzheimer's disease (2010-2050). Alzheimers Dement. March 2014; 10(2):e40-46.
22. James B D, Leurgans S E, Hebert L E, Scherr P A, Yaffe K, Bennett D A. Contribution of Alzheimer disease to mortality in the United States. Neurology Mar. 25 2014; 82(12):1045-1050.
23. Reddy P H, Tonk S, Kumar S, et al. A critical evaluation of neuroprotective and neurodegenerative MicroRNAs in Alzheimer's disease. Biochem Biophys Res Commun. Feb. 19 2017; 483(4):1156-1165.
24. Villemagne V L, Dore V, Bourgeat P, et al. Abeta-amyloid and Tau Imaging in Dementia. Semin Nucl Med. January 2017; 47(1):75-88.
25. Robertson J S, Rowe C C, Villemagne V L. Tau imaging with PET: an overview of challenges, current progress, and future applications. QJ Nucl Med Mol Imaging. December 2017; 61(4):405-413.
26. Blennow K, Hampel H, Weiner M, Zetterberg H. Cerebrospinal fluid and plasma biomarkers in Alzheimer disease Nat Rev Neurol. March 2010; 6(3):131-144.
27. Marksteiner J, Hinterhuber H, Humpel C. Cerebrospinal fluid biomarkers for diagnosis of Alzheimer's disease: beta-amyloid(1-42), tau, phospho-tau-181 and total protein. Drugs Today (Barc). June 2007; 43(6):423-431.
28. Perez-Grijalba V, Pesini P, Allue I A, et al. Abeta1-17 is a major amyloid-beta fragment isoform in cerebrospinal fluid and blood with possible diagnostic value in Alzheimer's disease. J Alzheimers Dis. 2015; 43(1):47-56.
29. Holmes C, Cunningham C, Zotova E, et al. Systemic inflammation and disease progression in Alzheimer disease. Neurology. Sep. 8 2009; 73(10):768-774.
30. Laske C, Schmohl M, Leyhe T, et Immune profiling in blood identifies sTNF-R: performing comparably as biomarker panels for classification of Alzheimer's disease patients. J Alzheimers Dis. 2013; 34(2):367-375.
31. Mastrangelo M A, Sudol K L. Narrow W C, Bowers W J. Interferon-(gamma) differentially affects Alzheimer's disease pathologies and induces neurogenesis in triple transgenic-AD mice. Am J Pathol. November 2009; 175(5):2076-2088.
32. Tobinick E. Tumour necrosis factor modulation for treatment of Alzheimer's disease: rationale and current evidence. CNS Drugs. September 2009; 23(9):713-725.
33. Koyama A, Okereke O I, Yang T, Blacker D, Selkoe D J, Grodstein F. Plasma amyloid-beta predictor of dementia and cognitive decline: a systematic review I meta-analysis. Arch Neurol. 12012; 69(7):824-831.
34. Geekiyanage H, Jicha G A, Nelson P T, Chan C. Blood serum miRNA: non-invasive biomarkers for Alzheimer's disease. Exp Neurol. June 2012; 235(2):491-496.
35. Schipper H M, Maes O C, Chertkow H M, Wang E. MicroRNA expression in Alzheimer blood mononuclear cells. Gene Regul Syst Bio. Dec. 20 2007; 1:263-274.
36. Villa C, Ridolfi E, Fenoglio C, et al. Expression of the transcription factor Sp1 and its regulatory hsa-miR-29b in peripheral blood mononuclear cells from patients with Alzheimer's disease. J Alzheimers Dis. 2013; 35(3):487-494.
37. Drummond M J, Rasmussen B B. Leucine-enriched nutrients and the regulation of mammalian target of rapamycin signalling and human skeletal muscle protein synthesis. Curr Opin Clin Nutr Metab Care. May 2008; 11(3):222-226.
38. Kreider R B. Dietary supplements and the promotion of muscle growth with resistance exercise. Sports Med. February 1999; 27(2):97-110.
39. Layman C leucine in protein metabolism during exercise recovery. Appl Physiol. 2002; 27(6):646-663.
40. Bower R H, Muggia-Sullam, Vallgren S, et al. Branched chain amino acid-enriched solutions in the septic patient. A randomized, prospective trial. Ann Surg. January 1986; 203(1):13-20.
41. Chin S E, Shepherd R W, Thomas B., et al. Nutritional support in children with end-stage liver disease: randomized crossover trial of a branched-chain amino acid supplement. Am J Clin Nutr. July 1992; 56(1):158-163.
42. Choudry H A, Pan M, Karinch A M, Souba W W. Branched-chain amino acid-enriched nutritional support in surgical and cancer patients. /Nutr. January 2006; 136(1 Suppl):3145-3185.
43. Habu D, Nishiguchi S, Nakatani Effect of 1 supplementation with branched-chain d granules on serum albumin level in the early stage of cirrhosis: a randomized pilot trial. Hepatol Res. March 2003; 25(3):312-318.
44. Ishihara T, Iwasa M, Tanaka H, et al. Effect of branched-chain amino acids in patients receiving intervention for hepatocellular carcinoma. World/Gastroenterol. Mar. 14 2014; 20(10):2673-2680.
45. Kakazu Kondo Y, Kogure et al. Supplementation of branched-chain amino acids maintains the albumin level in the course of hepatocellular carcinoma recurrence. Tohoku J Exp Med. 2013; 230(4):191-196.
46. Matsuoka Tamura A, Nakagawara H, Moriyama M. Improvement in the nutritional status and clinical conditions of patients with liver failure using liver diet combined with: branched amino acids-enriched elemental diet. Hepatogastroenterology. July-August 2014; 61(133):1308-1312.
47. Tietze I N, Pedersen E B. Effect of fish protein supplementation on aminoacid profile and nutritional status in haemodialysis patients. Nephrol Dial Transplant. 1991; 6(12):948-954.
48. Togo S, Tanaka K, Morioka D, et al. Usefulness of granular BCAA after hepatectomy for liver cancer complicated with liver cirrhosis. Nutrition. April 2005; 21(4):480-486.
49. Tsien C, Davuluri G, Singh D, et al. Metabolic and molecular responses to leucine-enriched branched chain amino acid supplementation in the skeletal muscle of alcoholic cirrhosis. Hepatology. June 2015; 61(6):2018-2029.
50. Amaral A U, Leipnitz Fernandes, Seminotti B, Schuck P F, Wajner Alpha-ketoisocaproic acid and leucine provoke mitochondrial bioenergetic dysfunction in rat brain. Brain Res. Apr. 9 2010; 1324:75-84.
51. Lu G, Ren S, Korge P, et al. A novel mitochondrial matrix serine/threonine protein phosphatase regulates the mitochondria permeability transition pore and is essential for cellular survival development. Genes Dev. April 12007; 21(7):784-796.
52. syrup Haymond M W, Ben-Galim E, Strobel K E. Glucose and alanine metabolism in children with maple disease. J Clin Invest. August 1978; 62(2):398-405.
53. Snyderman S E, Goldstein F, Sansaricg C, Norton P M. The relationship between the branched chain amino acids and their alpha-ketoacids in maple syrup urine disease. Pediatr I September 1984; 18(9):851-853.
54. Brosnan J T, Brosnan M E. Branched-chain amino acids: enzyme and substrate regulation. J Nutr. January 2006; 136(1 Suppl):207S-211 S.
55. Jeganathan S, Abdullahi A, Zargar S, Maeda N, Riddell M C, Adegoke O A. Amino acid-induced impairment of insulin sensitivity in healthy and obese rats is reversible. Physiol Rep. Jul. 1 2014; 2(7).
56. Li Yin Tan Kong X, Wu G. Leucine nutrition in animals and humans: mTOR signaling and beyond. Amino Acids. November 2011; 41(5):1185-1193.
57. Adams S H, Hoppel C L, Lok K H, et al. Plasma acylcarnitine profiles suggest incomplete long-chain fatty acid beta-oxidation I altered tricarboxylic acid cycle activity in type diabetic African-American Nutr. June 2009; 139(6):1073-1081.
58. Kim J Y, Park J Y, Kim O Y, et al. Metabolic profiling of plasma in overweight/obese and lean men using ultra performance liquid chromatography and Q-TOF mass spectrometry (UPLC-Q-TOF MS). Proteome Res. September 2010; 9(9):4368-4375.
59. Mihalik S J, Goodpaster B H, Kelley D E, et al. Increased levels of plasma acylcarnitines in obesity and type diabetes and identification of a marker of glucolipotoxicity. Obesity (Silver Spring). September 2010; 18(9):1695-1700.
60. Larsson S C, Markus H S. Branched-chain amino acids and Alzheimer's disease: a Mendelian randomization analysis. Sci Rep. Oct. 19 2017; 7(1):13604.
61. Toledo J B, Arnold M, Kastenmuller G, et al. Metabolic network failures in Alzheimer's disease: A biochemical road map. Alzheimers Dement. September 2017; 13(9):965-984.
62. Tynkkynen J, Chouraki V, van der Lee S J, et al. Association of branched-chain amino acids and other circulating metabolites with risk of incident dementia Alzheimer's disease: A prospective study eight cohorts. Alzheimers Dement. June 2018; 14(6):723-733.
63. Kobayashi R, Shimomura Y, Murakami T, et al. Gender difference in regulation of branched-chain amino acid catabolism. Biochem J. 1997; 327 Pt 2):449-453.
64. Obayashi M, Shimomura Y, Nakai N, et al. Estrogen controls branched-chain amino acid catabolism in female rats. /Nutr. October 2004; 134(10):2628-2633.
65. de Toledo Ferraz Alves T C, Ferreira L K, Wajngarten M, Busatto G F. Cardiac disorders as risk factors for Alzheimer's disease. J Alzheimers Dis. 2010; 20(3):749-763.
66. Santos C Y, Snyder P J, Wu W C, Zhang M, Echeverria A, Alber J. Pathophysiologic relationship between Alzheimer's disease, cerebrovascular disease, and cardiovascular risk: A review and synthesis. Alzheimers Dement (Amst). 2017; 7:69-87.
67. Li H, Ye D. Xie W, et al. Defect of branched-chain amino acid metabolism promotes the development of Alzheimer's disease by targeting the mTOR signaling. Biosci Rep. Aug. 31 2018; 38(4).
68. Ruiz H H, Chi Shin A C, I. Increased susceptibility o metabolic dysregulation in model of Alzheimer's disease is associated with impaired hypothalamic insulin signaling and elevated BCAA levels. Alzheimers Dement. August 2016; 12(8):851-861.
69. Macklin Griffith C M, Rose Patrylo P R. Glucose tolerance and insulin sensitivity are impaired in APP/PS1 transgenic mice prior to amyloid plaque pathogenesis and cognitive decline. Exp Gerontol. February 2017; 88:9-18.
70. Radde R, Bolmont T, Kaeser S A, et al. Abeta42-driven cerebral amyloidosis in transgenic mice reveals early and robust pathology. EMBO Rep. September 2006; 7(9):940-946.
71. Serneels L, Van Biervliet J, Craessaerts K, et al. gamma-Secretase heterogeneity in the Aph1 subunit: relevance for Alzheimer's disease. Science. May 1 2009; 324(5927):639-642.
72. Deacon R M, Rawlins J N. T-maze alternation in the rodent. Nat Protoc. 2006; 1(1):7-12.
73. Stewart S. Cacucci F. Lever C. Which memory task for my mouse? A systematic review of spatial memory performance in the Tg2576 Alzheimer's mouse model. J Alzheimers Dis. 2011; 26(1):105-126.
74. Morris R. Developments of a water-maze procedure for studying spatial learning in the rat. J Neurosci Methods. May 1984; 11(1):47-60.
75. Hegde V, Vijayan M, Kumar 5, et al. Adenovirus 36 improves glycemic control and markers of Alzh pathogenesis. Biochim Biophys Acta Mol Basis Dis. Nov. 1 2019; 1865(11):165531.
76. Patkar P P, Z, Mumphrey M B, Townsend R L, Berthoud H R, Shin A C. Unlike calorie restriction, Roux-en-Y gastric bypass surgery does not increase hypothalamic AgRP and NPY in mice on a high-fat diet. Int J Obes (Lond). Feb. 4 2019.
77. Shin A C, MohanKumar S M, Sirivelu M P, et al. Chronic exposure high-fat diet affects stress axis function differentially in diet-induced obese and diet-resistant rats. Int J Obes (Lond). July 2010; 34(7):1218-1226.
78. Shin A C, Pistell P J, Phifer C B, Berthoud H R. Reversible suppression of food reward behavior by chronic mu-opioid receptor antagonism in the nucleus accumbens. Neuroscience. Oct. 3 2010; 170(2):580-588.
79. Beckett P R. Spectrophotometric assay for measuring branched-chain amino acids. Methods Enzymol. 2000; 324:40-47.
80. She P Van Horn C, Reid T, Hutson S M, Cooney R N, Lynch C J. Obesity-related elevations in plasma leucine are associated with alterations in enzymes involved in branched-chain amino acid metabolism. Am J Physiol Endocrinol Metab. December 2007; 293(6):E1552-1563.
81. Shin A C Zheng H, Pistell P J, Berthoud H R. Roux-en-Y gastric bypass surgery changes food reward in rats. Int J Obes (Lond). May 2011; 35(5):642-651.
82. Shin A C, Townsend R L, Patterson L M, Berthoud H R. “Liking” and “wanting” of sweet and oily food stimuli as affected by high-fat diet-induced obesity, weight loss, leptin, and genetic predisposition. Am J Physiol Regul Integr Comp Physiol. November 2011; 301(5):R1267-1280.
83. Liu P, Ji Y, Yuen T, et al. Blocking FSH induces thermogenic adipose tissue and reduces body fat. Nature. Jun. 1 2017; 546(7656):107-112.
84. Hutson S M, Sweatt A J, Lanoue K F. Branched-chain [corrected] amino acid metabolism: implications for establishing safe intakes. J Nutr. June 2005; 135(6 Suppl):1557S-1564S.
85. Suryawan A, Hawes J W, Harris R A, Shimomura Y, Jenkins A E, Hutson S M. A molecular model of human branched-chain amino acid metabolism. Am J Clin Nutr. July 1998; 68(1):72-81.
86 Shin A C, Filatova N, Lindtner C, Insulin Receptor Signaling in POMC, but Not AgRP, Neurons Controls Adipose Tissue Insulin Action. Diabetes. June 2017; 66(6):1560-1571.
87. Lin Y S, Lin F Y, Hsiao H. Myostatin Is Associated With Cognitive Decline in an Animal Model of Alzheimer's Disease. Mol Neurobiol. March 2019; 56(3):1984-1991.
88. Parrella E, Maxim T, Maialetti F, et al. Protein restriction cycles reduce IGF-1 and phosphorylated Tau, and improve behavioral performance in an Alzheimer's disease mouse model. Aging Cell. April 2013; 12(2):257-268.
89. Gratuze M, Julien J, Morin F, Marette A, Planel Differential effects of voluntary treadmill exercise and caloric restriction on tau pathogenesis in a mouse model of Alzheimer's disease-like tau pathology fed with Western diet. Prog Neuropsychopharmacol Biol Psychiatry. October: 2017; 79(Pt B):452-461.
90. Sanchez-Roman I, Barja G. Regulation of longevity and oxidative stress by nutritional interventions: role of methionine restriction. Exp Gerontol. October 2013; 48(10):1030-1042.
91. Wang R, Dong W, Zhao Z. Caloric restriction can improve learning and memory in C57/BL mice probably via regulation of the AMPK signaling pathway. Exp Gerontol. February 2018; 102:28-35.
92. Tournissac M, Vandal M, Tremblay C, et al. Dietary intake of branched-chain amino acids model of Alzheimer's disease: Effects on survival, behavior, and neuropathology. Alzheimers Dement (N Y). 2018; 4:677-687.
93 Stover K R, Campbell M A, Van Winssen C M, Brown R E. Early detection of cognitive deficits the 3xTg-AD model of Alzheimer's disease. Behav Brain Res. Aug. 1 2015; 289:29-38.
94. White P J, Lapworth A L, An J, et al. Branched-chain amino acid restriction in Zucker-fatty rats improves muscle insulin sensitivity by enhancing efficiency of fatty acid oxidation and acyl-glycine export. Mol Metab. July 2016; 5(7):538-551.

1. A method of treating CNS-related conditions, comprising:
administering an effective amount of a compound that lowers the levels of one or more branched-chain amino acids (BCAAs) or metabolites thereof, or any pharmaceutically acceptable salt thereof, wherein the CNS-related conditions is selected from the group consisting of Alzheimer's disease and dementia selected from the group consisting of vascular dementia, Lewy body dementia, frontotemporal dementia, Creutzfeldt-Jacob disease, Wernicke-Korsakoff disease, and Huntington's disease, Multiple sclerosis, Parkinson's disease, autism, Amyotrophic lateral sclerosis (ALS), Hereditary diffuse leukoencephalopathy with spheroids (HDLS) and epilepsy, neuropsychiatric disorders, generalized anxiety disorder, social anxiety disorder, specific phobias and separation anxiety disorder, depression disorders, clinical depression (major depression), bipolar depression, persistent depressive disorder (dysthymia), seasonal affective disorder, atypical depression, treatment-resistant depression, psychotic depression, postpartum depression, premenstrual dysphoric disorder, and situational depression (stress response syndrome).

2. The method of claim 1, wherein the compound is a (S)-α-chloro-phenylpropionic acid and is administered in an amount within a range of from 10-200 mg/kg and is provided daily.

3. The method of claim 1, wherein the compound is a BT2 compound and is provided at administered in an amount within a range of from 10-100 mg/kg/day and is provided daily.

4. The method of claim 3, wherein the BT2 compounds is selected from at least one of: BT2 (3,6-dichlorobenzo[b]thiophene-2-carboxylic acid); BT2F (3-chloro-6-fluorobenzo[b]thiophene-2-carboxylic acid); or BT3 (N-(4-acetamido-1,2,5-oxadiazol-3-yl)-3,6-dichlorobenzo[b]thiophene-2-carboxamide).

5. The method of claim 1, wherein the compound is a benzothiopene-2-carboxylic acid (BT2) compound and is provided at administered in an amount within a range of from 10-100 mg/kg/day and is provided daily.

6. The method of claim 1, wherein the compound is administered using a method selected from the group consisting of oral, intravenous, subcutaneous, intranasal, pulmonary, intracranial, intracerebroventricular, topical, enteral, parenteral, or rectal.

7. The method of claim 1, wherein the compound is administered in a formulation comprising a carrier, the carrier being selected from the group consisting of: lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil.

8. The method of claim 1, wherein the CNS-related condition treated by the compound is Alzheimer's Disease.

9. The method of claim 1, further comprising obtaining a biological sample from the patient with the CNS-related condition and determining if the biological sample has an increase in BCAAs or metabolites thereof when compared to a sample from a subject without the CNS-related condition.

10. A method of identifying and treating CNS-related conditions, comprising:
obtaining a biological sample from the patient with the CNS-related condition and determining if the biological sample has an increase in one or more branched-chain amino acids (BCAAs) or metabolites thereof, when compared to a sample from a subject without the CNS-related condition; and
administering an effective amount of a compound that lowers the levels of one or more branched-chain amino acids (BCAAs) or metabolites thereof, or any pharmaceutically acceptable salt thereof, wherein the CNS-related conditions is selected from the group consisting of Alzheimer's disease and dementia selected from the group consisting of vascular dementia, Lewy body dementia, frontotemporal dementia, Creutzfeldt-Jacob disease, Wernicke-Korsakoff disease, and Huntington's disease, Multiple sclerosis, Parkinson's disease, autism, Amyotrophic lateral sclerosis (ALS), Hereditary diffuse leukoencephalopathy with spheroids (HDLS) and epilepsy, neuropsychiatric disorders, generalized anxiety disorder, social anxiety disorder, specific phobias and separation anxiety disorder, depression disorders, clinical depression (major depression), bipolar depression, persistent depressive disorder (dysthymia), seasonal affective disorder, atypical depression, treatment-resistant depression, psychotic depression, postpartum depression, premenstrual dysphoric disorder, and situational depression (stress response syndrome).

11. The method of claim 10, wherein the compound is a (S)-α-chloro-phenylpropionic acid and is administered in an amount within a range of from 10-200 mg/kg and is provided daily; or
the compound is a benzothiopene-2-carboxylic acid (BT2) compound and is provided at administered in an amount within a range of from 10-100 mg/kg/day and is provided daily; or
the BT2 compounds is selected from at least one of: BT2 (3,6-dichlorobenzo[b]thiophene-2-carboxylic acid); BT2F (3-chloro-6-fluorobenzo[b]thiophene-2-carboxylic acid); or BT3 (N-(4-acetamido-1,2,5-oxadiazol-3-yl)-3,6-dichlorobenzo[b]thiophene-2-carboxamide); or
the compound is a BT2 compound and is provided at administered in an amount within a range of from 10-100 mg/kg/day and is provided daily; and
wherein the compound is administered using a method selected from the group consisting of oral, intravenous, subcutaneous, intranasal, pulmonary, intracranial, intracerebroventricular, topical, enteral, parenteral, or rectal.

12. (canceled)

13. (canceled)

14. (canceled)

15. (canceled)

16. The method of claim 10, wherein the compound is administered in a formulation comprising a carrier, the carrier being selected from the group consisting of: lactose, dextrose, sucrose, sorbitol, mannitol, xylitol, erythritol, maltitol, starches, gum acacia, alginate, gelatin, calcium phosphate, calcium silicate, cellulose, methyl cellulose, microcrystalline cellulose, polyvinylpyrrolidone, water, methyl benzoate, propyl benzoate, talc, magnesium stearate, and mineral oil.

17. The method of claim 10, wherein the CNS-related condition treated by the compound is Alzheimer's Disease.

18. The method of claim 10, further comprising obtaining one or more additional biological samples at a different time from the patient with the CNS-related condition after treatment with the composition that lowers one or more BCAAs or metabolites thereof, and determining if the biological sample has an decrease in BCAAs when compared to a prior sample from the subject with the CNS-related condition.

19. A method for treating a patient with a CNS-related condition that comprises an increase in one or more branched-chain amino acids (BCAAs) or metabolites thereof, the method comprising the steps of:
performing or having performed an assay from a biological sample from the patient with the CNS-related condition to determine if the biological sample has an increase in one or more branched-chain amino acids (BCAAs) or metabolites thereof, when compared to a sample from a subject without the CNS-related condition; and
if the biological sample from the patient with the CNS-related condition has an increase in the one or more BCAAs or metabolites thereof, then:
treating the patient with an effective amount of a compound that lowers the levels of one or more branched-chain amino acids (BCAAs) or metabolites thereof, or any pharmaceutically acceptable salt thereof, wherein the CNS-related conditions is selected from the group consisting of Alzheimer's disease and dementia selected from the group consisting of vascular dementia, Lewy body dementia, frontotemporal dementia, Creutzfeldt-Jacob disease, Wernicke-Korsakoff disease, and Huntington's disease, Multiple sclerosis, Parkinson's disease, autism, Amyotrophic lateral sclerosis (ALS), Hereditary diffuse leukoencephalopathy with spheroids (HDLS) and epilepsy, neuropsychiatric disorders, generalized anxiety disorder, social anxiety disorder, specific phobias and separation anxiety disorder, depression disorders, clinical depression (major depression), bipolar depression, persistent depressive disorder (dysthymia), seasonal affective disorder, atypical depression, treatment-resistant depression, psychotic depression, postpartum depression, premenstrual dysphoric disorder, and situational depression (stress response syndrome).

20. The method of claim 19, wherein the compound is a (S)-α-chloro-phenylpropionic acid and is administered in an amount within a range of from 10-200 mg/kg and is provided daily; or
the compound is a benzothiopene-2-carboxylic acid (BT2) compound and is provided at administered in an amount within a range of from 10-100 mg/kg/day and is provided daily; or
the BT2 compounds is selected from at least one of: BT2 (3,6-dichlorobenzo[b]thiophene-2-carboxylic acid); BT2F (3-chloro-6-fluorobenzo[b]thiophene-2-carboxylic acid); or BT3 (N-(4-acetamido-1,2,5-oxadiazol-3-yl)-3,6-dichlorobenzo[b]thiophene-2-carboxamide); or
the compound is a BT2 compound and is provided at administered in an amount within a range of from 10-100 mg/kg/day and is provided daily; and
wherein the compound is administered using a method selected from the group consisting of oral, intravenous, subcutaneous, intranasal, pulmonary, intracranial, intracerebroventricular, topical, enteral, parenteral, or rectal.

21. (canceled)

22. (canceled)

23. (canceled)

24. (canceled)

25. A method for diagnosing a patient with a CNS-related condition related to increases in branched-chain amino acids (BCAAs) or metabolites thereof, the method comprising:
performing or having performed an assay from a biological sample from the patient with the CNS-related condition to determine if the biological sample has an increase in one or more branched-chain amino acids (BCAAs) or metabolites thereof, when compared to a sample from a subject without the CNS-related condition, wherein the BCAA is selected from at least one of valine, leucine, and isoleucine, or metabolites thereof.

26. The method of claim 25, wherein the biological sample is selected from blood, plasma, serum, tear, sweat, or sputum.

27. The method of claim 25, wherein the CNS-related condition is selected from at least one of: Alzheimer's disease and dementia selected from the group consisting of vascular dementia, Lewy body dementia, frontotemporal dementia, Creutzfeldt-Jacob disease, Wernicke-Korsakoff disease, and Huntington's disease, Multiple sclerosis, Parkinson's disease, autism, Amyotrophic lateral sclerosis (ALS), Hereditary diffuse leukoencephalopathy with spheroids (HDLS) and epilepsy, neuropsychiatric disorders, generalized anxiety disorder, social anxiety disorder, specific phobias and separation anxiety disorder, depression disorders, clinical depression (major depression), bipolar depression, persistent depressive disorder (dysthymia), seasonal affective disorder, atypical depression, treatment-resistant depression, psychotic depression, postpartum depression, premenstrual dysphoric disorder, and situational depression (stress response syndrome).
